(19) United States
(12) Patent Application Publication
(10) Pub. No.: US 2020/0165189 A1
(43) Pub. Date: May 28, 2020

(54) PSEUDO TRÖGER'S BASE AMINES AND MICROPOROUS POLYMERS DERIVED FROM PSEUDO TRÖGER'S BASE AMINES

(71) Applicant: King Abdullah University of Science and Technology, Thuwal (SA)

(72) Inventors: Mahmoud Atef ABDULHAMID, Thuwal (SA); Xiaohua MA, Thuwal (SA); Ingo PINNAU, Thuwal (SA)

(21) Appl. No.: 16/630,511

(22) PCT Filed: Jun. 14, 2018
(86) PCT No.: PCT/IB2018/054381

§ 371 Date: Jan. 13, 2020

Related U.S. Application Data
(60) Provisional application No. 62/531,456, filed on Jul. 12, 2017.

(51) Int. Cl. C07C 211/61; C07C 225/22; C08G 73/10; C08J 9/28; C08G 73/06; C08G 83/00; B01D 53/22; B01D 71/64

(52) U.S. Cl. CPC C07C 211/61; C07C 225/22; C08G 73/1067; C08J 9/28; C08G 73/0633; C08J 2201/05; C08G 73/1039; B01D 53/228; B01D 71/64; C07C 2603/88; C08J 2379/08; C08G 83/002

(57) ABSTRACT

Embodiments of the present disclosure describe carbocyclic pseudo Tröger's base (CTB) amines. Embodiments of the present disclosure further describe microporous polymers derived from pseudo CTB amines, including, but not limited to, polyimides, CTB ladder polymers, and network porous polymers. Other embodiments describe a method of separating chemical species in a fluid composition comprising contacting a microporous polymer membrane with a fluid composition including at least two chemical species, wherein the microporous polymer membrane includes one or more of a ladder polymer of intrinsic microporosity, a microporous polyimide, and a microporous network polymer; and capturing at least one of the chemical species from the fluid composition.

### BACKGROUND

At least one challenge to designing suitable microporous polymers for high-performing polymer-based gas separation membranes is that it is difficult to fabricate polymers that exhibit both high permeability and high selectivity. The empirical Robeson upper bound relationships define an inverse relationship between permeability and selectivity for polymeric membranes. For example, high permeability may be achieved at the cost of selectivity. One solution to overcoming this challenge and designing suitable microporous polymers is to achieve higher gas permeability by increasing the polymer's free volume (e.g., increased chain separation) and to achieve higher selectivity by increasing the polymer's rigidity.

Polymers of intrinsic microporosity (PIM) are one example of polymeric materials that possess high free volume due to contorted and rigid macromolecular chain architectures, which desirably promotes inefficient packing and chain rigidity, making them attractive for high-performing polymer-based gas separation membranes. Intrinsically microporous amorphous polymers have emerged as a burgeoning class of membrane materials with great potential in highly demanding gas separation applications. The microporous structure of PIMs results from the presence of highly rigid and contorted molecular building blocks, which severely restrain sufficient chain packing of the polymer matrix leading to high free volume.

The first generation of PIMs were based on ladder polymers derived from the reaction of 5,5′,6,6′-tetrahydroxy-3,3,3′,3′-tetramethyl-1,1′-spirobisindane and tetrafluoroterephtalonitrile (PIM-1) or with a 5,5′,6,6′-tetrachlorophenazyl-spirobisindane monomer (PIM-7). Recently developed ladder PIMs included using ethanoanthracene, triptycene, and Tröger's base building blocks.

The second generation of PIMs originated from extensions of earlier developments of low-free-volume polyimides that exhibited high selectivity but only low to moderate permeability. In 2008, a group reported for the first time the efficient incorporation of kinked spirobisindane contortion sites into polyimide structures to produce intrinsically microporous polyimides (PIM-PIs). PIM-PIs showed significantly higher gas permeability coupled with loss in gas-pair selectivity compared to conventional polyimides; however, their performance was close to the 2008 upper bounds for various gas pairs. Intensive investigations to tailor the structural design using ethanoanthracene- and 9,10-bridgehead-substituted triptycene moieties resulted in advanced PIM-PIs that demonstrated significantly enhanced selectivity for several gas pairs, especially O₂/N₂ and H₂/CH₄ while maintaining very high gas permeability. Moreover, hydroxyl- and carboxyl-functionalized PIM-PIs have shown excellent performance in removal of CO₂ and H₂S from methane in natural gas applications.

Later the same group reported ladder PIMs and PIM-PIs using Tröger's base-derived building blocks. Tröger's base is a chiral organic molecule, in which the chirality results from the presence of two bridgehead stereogenic nitrogen atoms in its structure. The cleft-like shape of Tröger's base, conferred by the diazocine bridge, resulted in incorporation of this rigid framework into some polymers with intrinsic microporosity. Tröger's base-derived PIM-PIs demonstrated good performance as materials for membrane-based gas separations with high permeabilities and commendable selectivities.

Recently, that group reported the synthesis of ladder PIMs derived from carbocyclic Tröger's base biscatechol analogues reacted with tetrafluoroterephtalonitrile. The corresponding PIMs displayed high BET surface area of up to 685 m² g⁻¹ but were either insoluble or had low molecular weight (M_{w}˜10,000 g mol⁻¹).

Accordingly, it would be desirable to provide building blocks for the synthesis of microporous polymers of high molecular weight and that are soluble in common organic solvents.

### SUMMARY

In general, embodiments of the present disclosure describe novel pseudo Tröger's base (TB) amines and polymers of intrinsic microporosity (PIM) based on PTB amines, as well as novel methods of making the pseudo TB amines and PIMs.

Accordingly, embodiments of the present disclosure describe a pseudo TB diamine characterized by the following chemical structure:

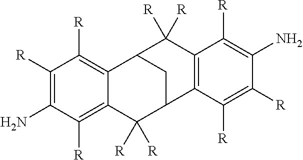

where each R is independently one or more of a hydrogen, a halogen and an alkyl group.

Embodiments of the present disclosure further describe a pseudo TB tetraamine characterized by the following chemical structure:

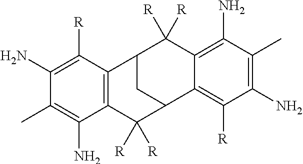

where each R is independently one or more of a hydrogen, a halogen and an alkyl group. Embodiments of the present disclosure also describe a polyimide characterized by the following chemical structure:

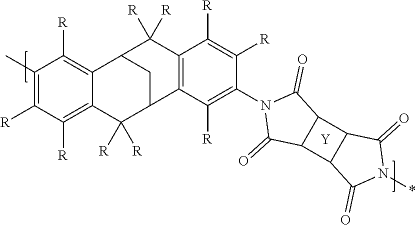

where Y is any dianhydride or multianhydride and each R is independently one or more of a hydrogen, a halogen and an alkyl group.

Another embodiment of the present disclosure is a Tröger's base ladder polymer characterized by the following chemical structure:

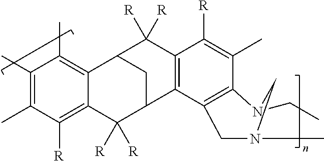

where each R is independently one or more of a hydrogen, a halogen and an alkyl group. Another embodiment of the present disclosure describes a network porous polymer characterized by the following chemical structure:

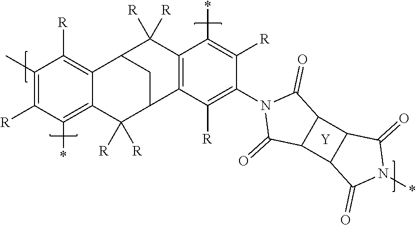

where Y is any dianhydride or multianhydride and each R is independently one or more of a hydrogen, a halogen and an alkyl group.

Another embodiment of the present disclosure describes a method of separating chemical species in a fluid composition comprising contacting a microporous polymer membrane with a fluid composition including at least two chemical species, wherein the microporous polymer membrane includes one or more of a ladder polymer of intrinsic microporosity, a microporous polyimide, and a microporous network polymer; and capturing at least one of the chemical species from the fluid composition.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of separating chemical species in a fluid composition, according to one or more embodiments of the present disclosure.

FIG. 2 is a flowchart of a method of synthesizing a pseudo TB, according to one or more embodiments of the present disclosure.

FIG. 3 is a flowchart of a method of synthesizing a pseudo TB amine, according to one or more embodiments of the present disclosure.

FIG. 4 is a flowchart of a method of fabricating a microporous polymer, according to one or more embodiments of the present disclosure.

FIG. 5 is a flowchart of a method of forming a ladder polymer of intrinsic microporosity, according to one or more embodiments of the present disclosure.

FIG. 6 is single-crystal XRD of intermediate dinitro compounds, according to one or more embodiments of the present disclosure.

FIG. 7 shows FT-IR spectra of 6FDA-CTBDA and 6FDA-iCTBDA polyimides, according to one or more embodiments of the present disclosure.

FIG. 8 is a graphical view of thermal gravimetric analysis (TGA) of 6FDA-CTBDA and 6FDA-iCTBDA polyimides, according to one or more embodiments of the present disclosure.

FIG. 9 illustrates nitrogen adsorption isotherms of 6FDA-CTBDA, 6FDA-iCTBDA at 77 K up to 1 bar, according to one or more embodiments of the present disclosure.

FIG. 10 is a graphical view of NLDFT-derived pore size distributions of 6FDA-CTBDA based on N₂ adsorption, according to one or more embodiments of the present disclosure.

FIG. 11 shows graphical views of CO₂ and CH₄ sorption isotherms measured gravimetrically at 35° C. for 6FDA-CTBDA according to one or more embodiments of the present disclosure

### DETAILED DESCRIPTION

The invention of the present disclosure relates to carbocyclic pseudo Tröger's base (CTB) amines, microporous polymers derived from the pseudo TB amines, and methods of synthesizing the pseudo TB amines and microporous polymers. The pseudo TB amines include carbocyclic pseudo TB diamine monomers and carbocyclic pseudo TB tetraamine monomers. These carbocyclic pseudo TB diamine and tetraamine monomers may react with various dianhydrides and/or multianhydrides to form a variety of microporous polymers and polymers of intrinsic microporosity (PIM). For example, the pseudo TB amine monomers may be used to form microporous polyimides, ladder polymers of intrinsic microporosity, and microporous network polymers. The microporous polymers are soluble in a wide variety of solvents, exhibit excellent chemical and thermal stability, and have high BET surface areas. In addition, the microporous polymers may be prepared via simple and efficient synthetic routes and exhibit excellent gas transport properties. In this way, the invention of the present disclosure provides novel pseudo TB amines and microporous polymers suitable for a wide variety of applications, including, but not limited to, membrane-based gas separations, aerospace industry, sensors for trace substance detection, electronic industry, and high-temperature adhesion and composite materials.

As one example, the invention of the present disclosure relates to a newly designed carbocyclic pseudo Tröger's base (TB) diamine monomer, 2,8-dimethyl-3,9-diamino-5,6,11,12-tetrahydro-5,11-methanodibenzo[a,e][8]annulene (CTBDA) and its isomeric analogue 2,8-dimethyl-(1,7)(4,10)(3,9)-diamino-5,6,11,12-tetrahydro-5,11-methanodibenzo[a,e][8]annulene (iCTBDA), which were used for the synthesis of intrinsically microporous 6FDA-based polyimides (6FDA-CTBDA and 6FDA-iCTBDA). Both polyimides were soluble in a wide variety of solvents, exhibited excellent thermal stability with decomposition temperature (T^{d,5%}) of ˜475° C., and had high BET surface areas of 587 m² g⁻¹ (6FDA-CTBDA) and 562 m² g⁻¹ (6FDA-iCTBDA).

### Definitions

The terms recited below have been defined as described below. All other terms and phrases in this disclosure shall be construed according to their ordinary meaning as understood by one of skill in the art.

As used herein, “anhydride” refers to a moiety of the formula R₁—C(═O)—O—C(═O)—R₂, where R₁ and R₂ are independently alkyl, haloalkyl, aryl, heteroaryl, cycloalkyl, aromatic alkyl, (cycloalkyl)alkyl and the like.

As used herein, “aryl” refers to a monovalent mono-, bi- or tricyclic aromatic hydrocarbon moiety of 6 to 15 ring atoms, which is optionally substituted with one or more, typically one, two, or three substituents within the ring structure. When two or more substituents are present in an aryl group, each substituent is independently selected. Exemplary aryl includes, but is not limited to, phenyl, 1-naphthyl, and 2-naphthyl, and the like, each of which can optionally be substituted.

As used herein, “alkyl group” refers to a functional group including any alkane with a hydrogen removed therefrom. For example, “alkyl” may refer to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl groups include, but are not limited to, methyl, ethyl, 1-propyl, 2-propyl, tert-butyl, pentyl, and the like.

As used herein, “capturing” refers to the act of removing one or more chemical species from a bulk fluid composition (e.g., gas/vapor, liquid, and/or solid). For example, “capturing” may include, but is not limited to, interacting, bonding, diffusing, adsorbing, absorbing, reacting, and sieving, whether chemically, electronically, electrostatically, physically, or kinetically driven.

As used herein, “carbocyclic” refers to a cyclic arrangement of carbon atoms forming a ring. The term “carbocyclic” may be distinguished from heterocyclic rings in which the ring backbone contains at least one atom which is different from carbon.

As used herein, “contacting” may refer to, among other things, feeding, flowing, passing, injecting, introducing, and/or providing the fluid composition (e.g., a feed gas).

As used herein, “halogen” refers to any elements classified as halogens according to the Periodic Table. Halogens may include one or more of fluorine, chlorine, bromine, and iodine.

As used herein, “heteroaryl group” refers to a monovalent mono- or bicyclic aromatic moiety of 5 to 12 ring atoms containing one, two, or three ring heteroatoms selected from N, O, or S, the remaining ring atoms being C. The heteroaryl ring can be optionally substituted with one or more substituents, typically one or two substituents. Exemplary heteroaryl includes, but is not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

As used herein, “microporous polymer” refers to one or more of polyimides (e.g., microporous polyimide), TB ladder polymers (e.g., ladder polymers of intrinsic microporosity), network porous polymers (e.g., microporous network polymer).

### Pseudo TB Amines

Embodiments of the present disclosure relate to, among other things, novel pseudo TB amines. In particular, embodiments of the present disclosure describe, among other things, pseudo TB diamine monomers. In many embodiments, the pseudo TB diamine monomer is a carbocyclic pseudo TB diamine monomer. For example, the carbocyclic pseudo TB diamine monomer may be characterized by the following chemical structure:

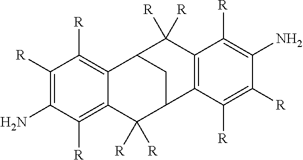

where each R is independently one or more of a hydrogen, a halogen and an alkyl group.

Each functional group (R) may be independently one or more of a hydrogen, a halogen and an alkyl group. The halogen may include one or more of fluorine, chlorine, bromine, and iodine. The alkyl group may include any alkyl group known in the art. The alkyl group may be cyclic or acyclic, aliphatic, linear or branched. In many embodiments, the alkyl group may include one or more of methyl, ethyl, propyl, isopropyl and iso-butyl.

In some embodiments, the carbocyclic pseudo TB diamine monomer may be characterized by one or more of the following chemical structures:

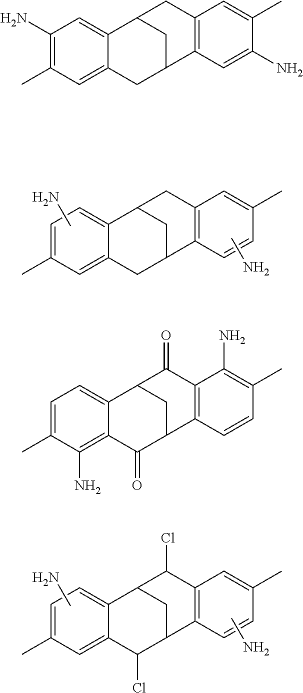

The carbocyclic pseudo TB diamine monomers may include any of the above monomers, as well as any of those monomers' isomeric analogues. For example, the carbocyclic pseudo TB diamine monomer may include 2,8-dimethyl-3,9-diamino-5,6,11,12-tetrahydro-5,11-methanodibenzo[a,e][8]annulene (CTBDA) and/or 2,8-dimethyl-(1,7)(4,10)(3,9)-diamino-5,6,11,12-tetrahydro-5,11-methanodibenzo[a,e][8]annulene (iCTBDA).

Embodiments of the present disclosure also describe, among other things, carbocyclic pseudo TB tetraamine monomers. In many embodiments, the pseudo TB tetraamine monomers is a carbocyclic pseudo TB tetraamine monomer. For example, the carbocyclic pseudo TB tetraamine monomer may be characterized by the following chemical structure:

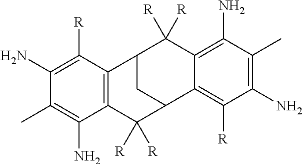

where each R is independently one or more of a hydrogen, a halogen and an alkyl group. Each functional group (R) may independently include any of the hydrogen, a halogen and an alkyl group of the present disclosure. In many embodiments, the functional groups (R) include any of those described with respect to the pseudo TB diamine monomer. Accordingly, that disclosure is incorporated by reference in its entirety here.

In some embodiments, the carbocyclic pseudo TB tetraamine monomer may be characterized by one or more of the following chemical structures:

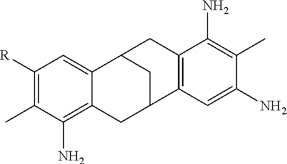

In particular, the carbocyclic pseudo TB tetraamine may be 2,8-dimethyl-5,6,11,12-tetrahydro-5,11-methanodibenzo[a,e][8]annulene-1,3,7,9-tetraamine. In addition, the carbocyclic pseudo TB diamine may include isomeric analogues of the above monomer.

### Polymer Materials

Embodiments of the present disclosure also relate to, among other things, various novel polymer materials, including, but not limited to, to polymers of intrinsic microporosity and microporous network polymers. For example, the polymer materials may include ladder polymers of intrinsic microporosity (PIM), microporous polyimides (PIM-PI), and microporous network polymers. Each of these polymer materials may be synthesized from any of the pseudo TB amine monomers disclosed herein and as described in greater detail below.

Accordingly, embodiments of the present disclosure describe microporous polyimides (PIM-PI). In many embodiments, the PIM-PIs may be characterized by the following chemical structure:

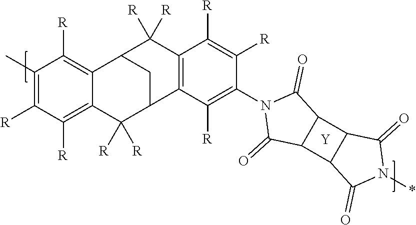

where Y is any anhydride—such as a dianhydride and/or multianhydride—and each R is independently one or more of a hydrogen, a halogen and an alkyl group. Each functional group (R) may independently include any of the hydrogen, a halogen and an alkyl group of the present disclosure. In many embodiments, the functional groups (R) include any of those described with respect to the pseudo TB diamine monomer. Accordingly, that disclosure is incorporated by reference in its entirety here.

The anhydride (Y) may be any dianhydride and/or multianhydride. The dianhydride and/or multianhydride may be one or more of aromatic, cycloaliphatic, and aliphatic. For example, the anyhydride may include a tetracarboxylic dianhydride, such as an aromatic tetracarboxylic dianhydride or a cyclaliphatic tetracarboxylic anhydride. In many embodiments, the anhydride (Y) may be characterized by one or more of the following chemical structures:

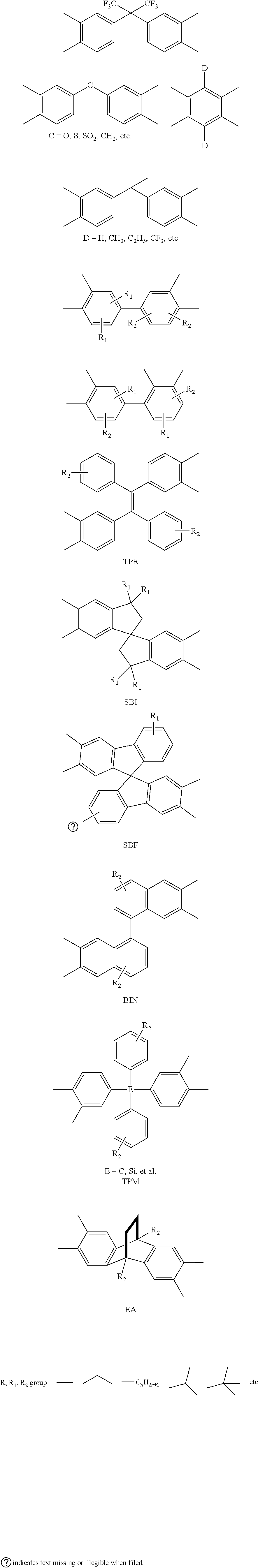

A suitable dianhydride must be chemical stable, contains at least one side of contortion and has some rigidity in its backbone structure.

Embodiments of the present disclosure also describe microporous network polymers. In many embodiments, the microporous network polymers may be characterized by the following chemical structure:

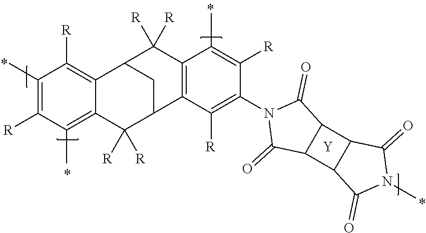

where Y is any anhydride—dianhydride and/or multianhydride—and each R is independently one or more of a hydrogen, a halogen and an alkyl group.

The anhydride (Y) may be any dianhydride and/or multianhydride. The dianhydride and/or multianhydride may be one or more of aromatic, cycloaliphatic, and aliphatic. In many embodiments, the anhydride (Y) may include any of the anhydrides disclosed above with respect to PIM-PI. Accordingly, the disclosure of anhydrides with respect to PIM-PI is hereby incorporated by reference in its entirety.

Each functional group (R) may independently include any of the hydrogen, a halogen and an alkyl group. of the present disclosure. In many embodiments, the functional groups (R) include any of those described with respect to the pseudo TB diamine monomer. Accordingly, that disclosure is incorporated by reference in its entirety here.

Embodiments of the present disclosure further describe ladder polymers of intrinsic microporosity (PIM). In many embodiments, the ladder polymer may be characterized by the following chemical structure:

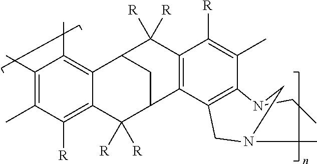

where each R is independently one or more of a hydrogen, a halogen and an alkyl group. Each functional group (R) may independently include any of the hydrogen, halogens, a halogen and an alkyl group of the present disclosure. In many embodiments, the functional groups (R) include any of those described with respect to the pseudo TB diamine monomer. Accordingly, that disclosure is incorporated by reference in its entirety here.

The microporous polymers—ladder polymers of intrinsic microporosity (PIM), microporous polyimides (PIM-PI), and microporous network polymers—of the present disclosure may be of high molecular weight with narrow polydispersity indexes. In many embodiments, the molecular weight of the polymers may range from about 150,000 g mol⁻¹ to about 170,000 g mol⁻¹ and the polydispersity index may range from about 1.6 to about 1.8. The microporous polymers may exhibit excellent solubility in common organic solvents, including, but not limited to, one or more of CHCl₃, THF, DMF, DMAc, NMP, and DMSO. In addition, the microporous polymers may exhibit high thermal stability with decomposition temperatures ranging from about 450° C. to about 490° C. The BET surface area of the microporous polymers range from about 550 m² g⁻¹ to about 590 m² g⁻¹ with pore size distributions ranging from about 7 Å or less to about 20 Å. In many embodiments, the pore size distribution of the microporous polymers include an ultra-microporous pore size of about 7 Å or less, with a significant fraction in the 10-20 Å range.

The microporous polymers may be used for membrane-based gas separation applications, among other things, including, but not limited to, air separation for nitrogen enrichment, hydrogen recovery from nitrogen and/or methane, as well as acid gas (CO₂/H₂S) removal and hydrocarbon recovery from natural gas streams. Further, these materials may be used for gas storage in aerospace, electronic industry applications, and in high temperature adhesion and composite materials. These applications shall not be limiting as the potential applications of these materials is unlimited.

### Methods of Separating Fluid Compositions

Membranes based on the microporous polymers of the present disclosure further exhibit gas transport properties. The ladder polymers of intrinsic microporosity, the microporous polyimides, and microporous network polymers may be used for membrane-based fluid separations. The microporous polymers exhibit high permeability and moderate to high selectivities. The fluids to be separated may be in any phase (e.g., gas/vapor, liquid, and/or solid) and may include a variety of chemical species. For example, the fluids to be separated may include at least O₂ and N₂, H₂ and N₂, H₂ and C₁₊ hydrocarbons, He and C₁₊ hydrocarbons, CO₂ and C₁₊ hydrocarbons, CO₂ and N₂, olefins and paraffins, n-butane and iso-butane, n-butane and butenes, xylene isomers, and combinations thereof. In many embodiments, the gas permeabilities of the microporous polymers followed the order H₂>CO₂>O₂>N₂>CH₄.

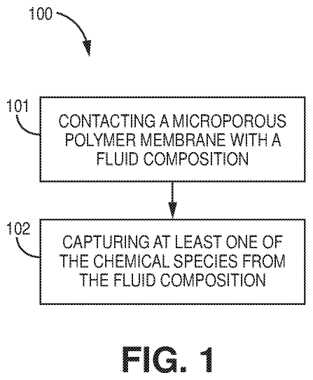

Accordingly, FIG. 1 is a flowchart of a method of separating chemical species in a fluid composition, according to one or more embodiments of the present disclosure. At step 101, a microporous polymer membrane is contacted with a fluid composition including at least two chemical species, wherein the microporous polymer membrane includes one or more of a ladder polymer of intrinsic microporosity, a microporous polyimide, and a microporous network polymer; wherein the ladder polymer of intrinsic porosity is characterized by the chemical structure:

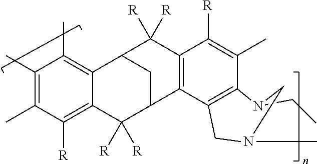

where each R is independently one or more of a hydrogen, a halogen and an alkyl group; wherein the microporous polyimide is characterized by the following chemical structure:

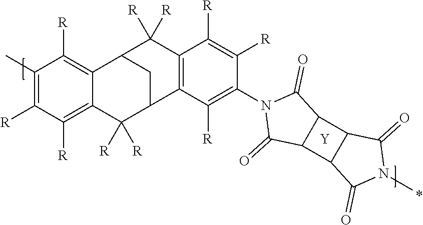

where Y is any anhydride—such as a dianhydride and/or multianhydride—and each R is independently one or more of a hydrogen, a halogen and an alkyl group; wherein the microporous network polymer is characterized by the following chemical structure:

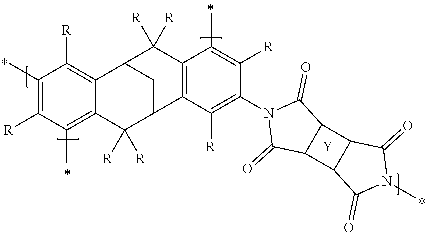

where Y is any anhydride—dianhydride and/or multianhydride—and each R is independently one or more of a hydrogen, a halogen and an alkyl group. At step 102, the microporous polymer membrane captures at least one of the chemical species from the fluid composition.

Contacting may refer to, among other things, feeding, flowing, passing, injecting, introducing, and/or providing the fluid composition (e.g., a feed gas). The contacting may occur at various pressures, temperatures, and concentrations of chemical species in the fluid composition, depending on desired feed conditions and/or reaction conditions. The pressure, temperature, and concentration at which the contacting occurred may be varied and/or adjusted according to a specific application.

The chemical species of the fluid composition may include one or more of O₂, N₂, H₂, He, CO₂, C₁₊ hydrocarbons, olefins, paraffins, n-butane, iso-butane, butenes, and xylene isomers. In many embodiments, the chemical species of the fluid composition may include at least one or more of the following pairs of chemical species: O₂ and N₂, H₂ and N₂, H₂ and C₁₊ hydrocarbons, He and C₁₊ hydrocarbons, CO₂ and C₁₊ hydrocarbons, CO₂ and N₂, olefins and paraffins, n-butane and iso-butane, n-butane and butenes, xylene isomers, and combinations thereof. In other embodiments, the chemical species of the fluid composition may include any combination of one or more of the chemical species described herein.

Capturing may refer to the act of removing one or more chemical species from a bulk fluid composition (e.g., gas/vapor, liquid, and/or solid). The capturing of the one or more chemical species may depend on a number of factors, including, but not limited to, selectivity, diffusivity, permeability, solubility, conditions (e.g., temperature, pressure, and concentration), membrane properties (e.g., pore size), and the methods used to fabricate the membranes.

The captured chemical species may include one or more of O₂, N₂, H₂, He, CO₂, C₁₊ hydrocarbons, olefins, paraffins, n-butane, iso-butane, butenes, and xylene isomers. In embodiments in which the fluid composition includes O₂ and N₂, the captured chemical species may include O₂. In embodiments in which the fluid composition includes H₂ and N₂, the captured chemical species may include H₂. In embodiments in which the fluid composition includes H₂ and C₁₊ hydrocarbons, the captured chemical species may include H₂. In embodiments in which the fluid composition includes He and C₁₊ hydrocarbons, the captured chemical species may include He. In embodiments in which the fluid composition includes CO₂ and C₁₊ hydrocarbons, the captured chemical species may include CO₂. In embodiments in which the fluid composition includes CO₂ and N₂, the captured chemical species may include CO₂. In embodiments in which the fluid composition includes olefins and paraffins, the captured chemical species may include olefins. In embodiments in which the fluid composition includes n-butane and iso-butane, the captured chemical species may include n-butane. In embodiments in which the fluid composition includes n-butane and butenes, the captured chemical species may include n-butane. These examples shall not be limiting, as in some embodiments, the captured species described above may be the non-captured species and the non-captured species described above may be the captured species.

### Methods of Synthesis

Embodiments of the present disclosure also relate to, among other things, methods of synthesizing the pseudo TB amines (e.g., the carbocyclic pseudo TB diamine monomers and the carbocyclic pseudo TB tetraamine monomers) and methods of forming polymer materials (e.g., PIM-PIs, microporous network polymers, and PIMs). In general, the polymer materials may be formed from the pseudo TB amines. For example, in many embodiments, the synthetic route may include one or more of the following steps in any order: (1) synthesizing a pseudo TB, (2) synthesizing a pseudo TB precursor, (3) synthesizing the pseudo TB amine, and (4) synthesizing the polymer material from the pseudo TB amine. A discussion of each of these synthetic routes, among others, is provided in greater detail below and elsewhere herein.

### Methods of Synthesizing Pseudo TB Amines

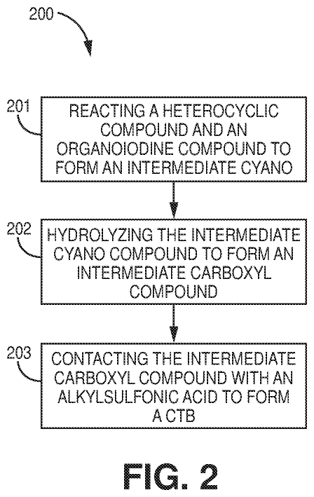

As shown in FIG. 2, a pseudo TB may be synthesized via a three-step synthetic route, according to one or more embodiments of the present disclosure. At step 201, a heterocyclic compound containing a cyano group is reacted with an organoiodine compound to form an intermediate cyano compound. At step 202, the intermediate cyano compound is hydrolyzed to form an intermediate carboxyl compound. At step 203, the intermediate carboxyl compound is contacted with an alkylsulfonic acid to form the pseudo TB.

Reacting the heterocyclic compound containing a cyano group with the organoiodine compound may include contacting in the presence of a strong base. In some embodiments, the reacting occurs at about 160° C. The strong base may include any strong base known in the art. In many embodiments, the strong base includes one or more of KOH and NaOH. In other embodiments, the strong base includes one or more of KOH, NaOH, K₂CO₃, Li₂CO₃.

The heterocyclic compound containing a cyano group may be characterized by the following chemical structure:

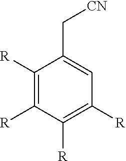

where each R is independently one or more of hydrogen, aliphatic alkyl groups, and halogen substituents. The aliphatic alkyl groups may include methyl, ethyl, propyl, isopropyl and iso-butyl. The halogen substituents may include one or more of bromine, chlorine, and fluorine. In many embodiments, the heterocyclic compound containing the cyano group is 2-phenylacetonitrile. In other embodiments, the heterocyclic compound containing the cyano group is 2-phenylacetonitrile. In many embodiments, the organoiodine compound is diiodomethane. The intermediate cyano compound (I) may be characterized by the following chemical structure:

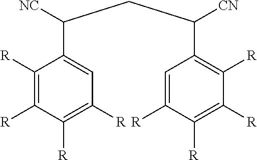

where each R is independently one or more of hydrogen, aliphatic alkyl groups and halogen substituents. The aliphatic alkyl groups and halogen substituents of the intermediate cyano compound may include any of the aliphatic alkyl groups and halogen substituents discussed above with respect to the heterocyclic compound containing a cyano group. Accordingly, that disclosure is hereby incorporated by reference in its entirety.

Hydrolyzing the intermediate cyano compound to form the intermediate carboxyl compound may include contacting with an aqueous solution including a strong base and/or an alcohol (e.g., ethanol)/water mixture including a strong base. In some embodiments, the hydrolyzing occurs at a temperature of about 100° C. The hydrolyzing step includes hydrolyzing cyano groups (—CN) to carboxylic acid groups (—COOH) to form the intermediate carboxyl compound.

The intermediate carboxyl compound (II) may be characterized by the following chemical structure:

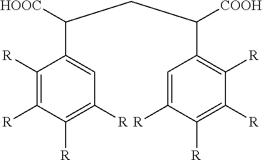

where each R is independently one or more of hydrogen, aliphatic alkyl groups and halogen substituents. The aliphatic alkyl groups and halogen substituents of the intermediate carboxyl compound may include any of the aliphatic alkyl groups and halogen substituents discussed above with respect to the heterocyclic compound containing a cyano group. Accordingly, that disclosure is hereby incorporated by reference in its entirety.

Contacting the intermediate carboxyl compound with the alkylsulfonic acid to form the pseudo TB may include mixing with the alkylsulfonic acid. In other embodiments, the contacting may include mixing, stirring, agitating, vibrating, and other methods of contacting known in the art. The alkylsulfonic acid may include any alkylsulfonic acid known in the art. In many embodiments, the alkylsulfonic acid is methanesulfonic acid.

The pseudo TB (III) may be characterized by the following chemical structure:

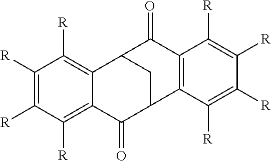

where R is one or more of hydrogen, aliphatic alkyl groups and halogen substituents. The aliphatic alkyl groups and halogen substituents of the pseudo TB may include any of the aliphatic alkyl groups and halogen substituents discussed above with respect to the heterocyclic compound containing a cyano group. Accordingly, that disclosure is hereby incorporated by reference in its entirety. In some embodiments, the pseudo TB is 5,11-methanodibenzo[a,e][8]annulene-6,12(5H, 11H)-dione pseudo TB.

In one embodiment, a pseudo TB may be synthesized according to the three-step synthetic route illustrated in Scheme 1:

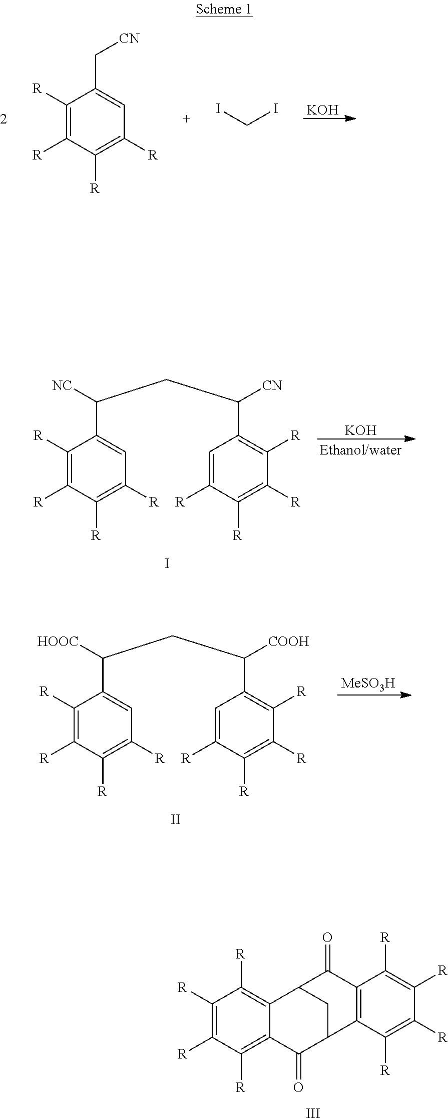

As shown in Scheme 1, the intermediate (I) is synthesized through a reaction between 2-phenylacetonitrile, where R is hydrogen, and diiodomethane in the presence of KOH at about 160° C. The intermediate carboxyl compound (II) is formed by hydrolyzing the cyano groups to carboxylic acid groups using KOH and a mixture of ethanol/water (1/1) at about 100° C. The desired pseudo TB is then prepared by mixing the intermediate carboxyl compound (II) with methanesulfonic acid at 80° C.

The pseudo TB may be used to form a pseudo TB precursor. The pseudo TB precursor may also be formed via a three-step synthetic route. In some embodiments, the three-step synthetic route includes reduction of the dione groups of the pseudo TB. For example, the three-step synthetic route for forming the pseudo TB precursor may be as shown in Scheme 2:

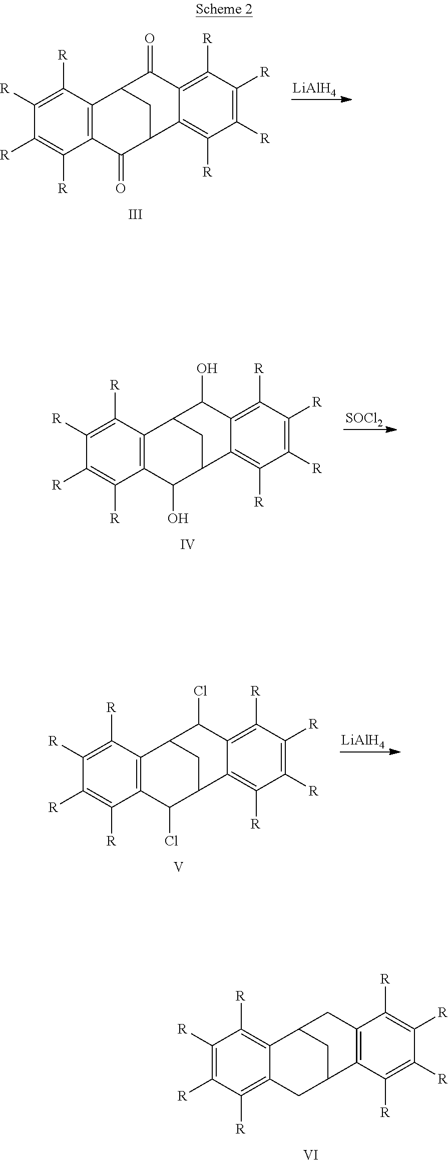

As shown in scheme 2, the carbonyl groups of the pseudo TB (III) may be converted to a hydroxyl groups to form a hydroxyl intermediate (IV) using, for example, lithium aluminum hydride (LiAlH₄) at about room temperature. Subsequently, the hydroxyl groups (—OH) of the hydroxyl intermediate (IV) may be replaced with chloro groups (—Cl) to form a chloro intermediate (V) by refluxing (e.g., overnight refluxing) with thionylchloride (SOCl₂), for example. Finally, the chloro groups of the chloro intermediate (V) may be replaced with hydrogens to form the pseudo TB precursor (VI) using, for example, lithium aluminum hydride at about 80° C. for about 12 hours.

In some embodiments, the synthetic route for forming a pseudo TB precursor may include replacing the carbonyl group of the pseudo TB (III) with other substituents. For example, a pseudo TB precursor with different substituents is shown in Scheme 3:

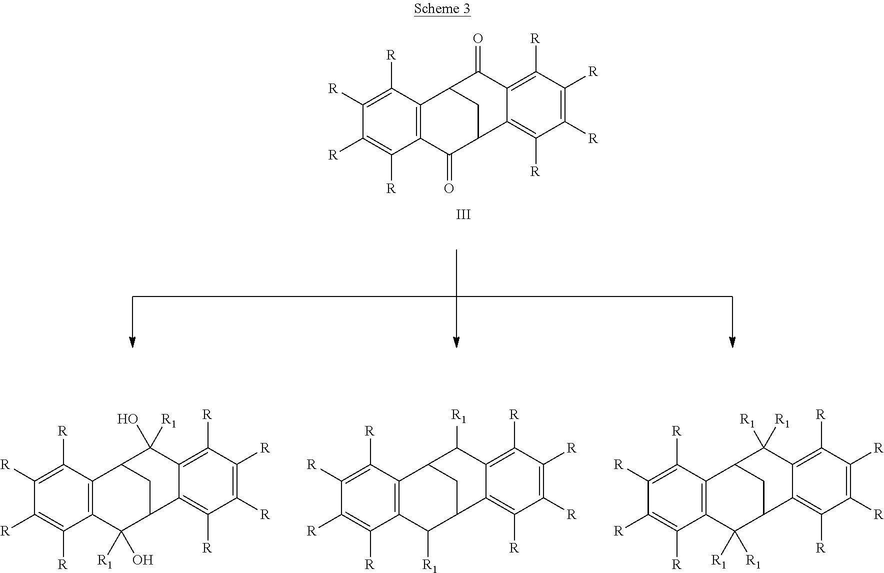

where each R and each R₁ are independently one or more of hydrogen, aliphatic alkyl groups and halogen substituents. The aliphatic alkyl groups and halogen substituents of the pseudo TB precursors may include any of the aliphatic alkyl groups, and halogen substituents discussed above with respect to the heterocyclic compound containing a cyano group. Accordingly, that disclosure is hereby incorporated by reference in its entirety.

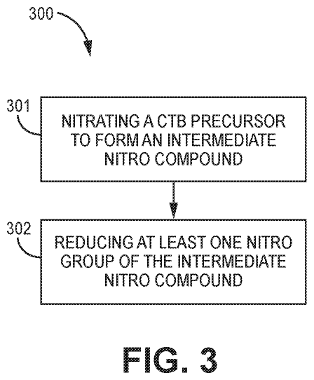

As shown in FIG. 3, a pseudo TB amine (e.g., carbocyclic pseudo TB amine) may be formed via a two-step synthetic route, according to one or more embodiments of the present disclosure. At step 301, a pseudo TB precursor is nitrated to form an intermediate nitro compound. At step 302, at least one nitro group of the intermediate nitro compound is reduced to form the pseudo TB amine.

Nitrating the pseudo TB precursor may include contacting with one or more of potassium nitrate (KNO₃), sulfuric acid (H₂SO₄), trifluoroacetic anhydride (TFAA), and nitric acid (HNO₃) to produce the intermediate nitro compound. In many embodiments relating to the synthesis of pseudo TB diamine monomers, nitrating the pseudo TB precursor includes contacting with potassium nitrate in a solution of either sulfuric acid or trifluoroacetic anhydride. In many embodiments relating to the synthesis of pseudo TB tetraamine monomers, nitrating the pseudo TB precursor includes contacting with nitric acid and sulfuric acid. In other embodiments, nitrating the pseudo TB precursor includes contacting with one or more of potassium nitrate (KNO₃), sulfuric acid (H₂SO₄), trifluoroacetic anhydride (TFAA), nitric acid (HNO₃).

The pseudo TB precursor may generally be characterized by one or more of the following chemical structures:

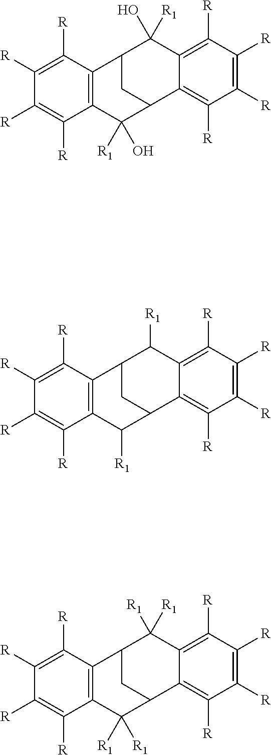

where R and R₁ is one or more of a hydrogen, a halogen and an alkyl group. The functional groups R and R₁ may include any of the hydrogen, halogens and aliphatic groups, discussed above with respect to the pseudo TB precursor. Accordingly, that disclosure is hereby incorporated by reference in its entirety.

In many embodiments, the pseudo TB precursor may include one or more of the following chemical structures:

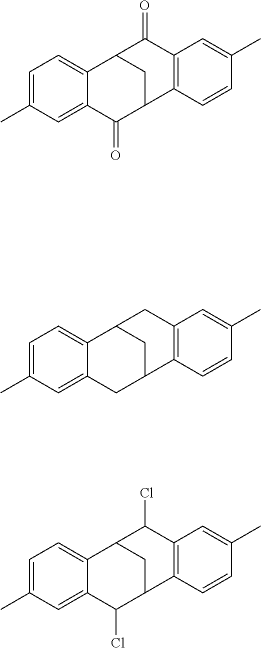

The intermediate nitro compound is formed by nitrating the pseudo TB precursor. In many embodiments, the intermediate nitro compound includes two nitro functional groups or four nitro functional groups. For example, in embodiments in which a pseudo TB diamine is formed, the intermediate nitro compound may include an intermediate dinitro compound. For example, the intermediate dinitro compound may be characterized by the following chemical structures:

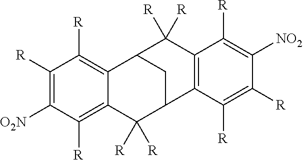

where each R is independently one or more of a hydrogen, a halogen and an aliphatic group. The functional groups R may include any of the hydrogen, halogens and aliphatic groups discussed above with respect to the pseudo TB precursor. Accordingly, that disclosure is hereby incorporated by reference in its entirety. In many embodiments, the intermediate dinitro compound may be characterized by one or more of the following chemical structures:

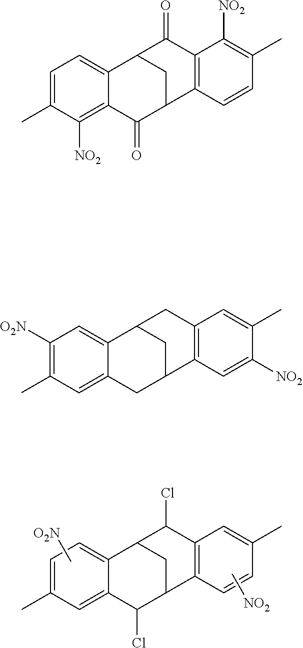

In embodiments in which a pseudo TB tetraamine is formed, the intermediate nitro compound may include an intermediate tetranitro compound. The intermediate tetranitro compound may be generally characterized by the following chemical structure:

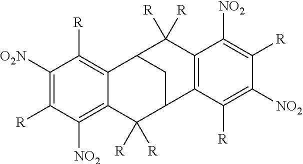

where each R is independently one or more of a hydrogen, a halogen and an aliphatic group. The functional groups R may include any of the hydrogen, halogens and aliphatic groups discussed above with respect to the pseudo TB precursor. Accordingly, that disclosure is hereby incorporated by reference in its entirety. In many embodiments, the intermediate tetranitro compound may be characterized by the following chemical structure:

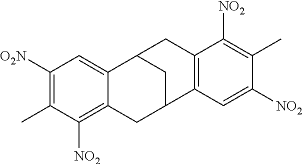 [SMILES: Cc1c([N+](=O)[O-])cc2c(c1[N+](=O)[O-])CC1CC2Cc2c1cc([N+](=O)[O-])c(C)c2[N+](=O)[O-]]

Reducing the at least one nitro group of the intermediate nitro compound may include replacing at least one nitro group of the intermediate nitro compound with an amine. Reducing may include reducing using one or more of hydrazine monohydrate (N₂H₄.H₂O) and palladium carbon (Pd/C) to achieve the amine. In embodiments in which a pseudo TB diamine is formed, reducing may include reducing two nitro groups of the intermediate nitro compound to amines. In embodiments in which a pseudo tetraamine is formed, reducing may include replacing four nitro groups of the intermediate nitro compound to amines.

In one embodiment, the pseudo TB diamine may generally be synthesized according to the synthetic route illustrated in Scheme 4:

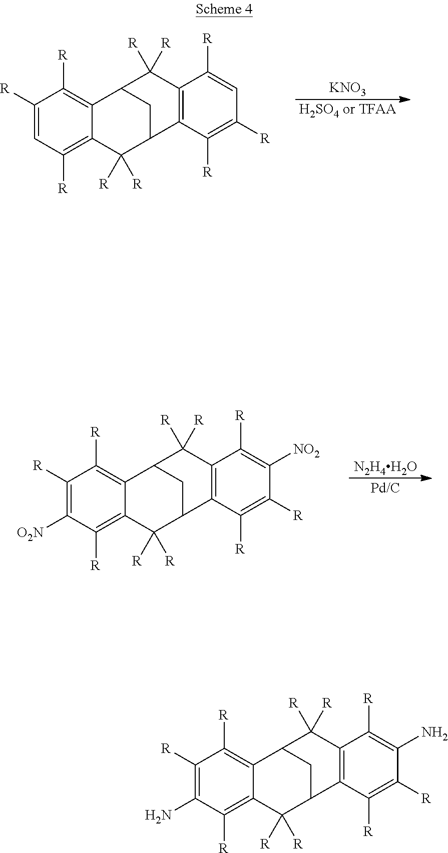

As shown in Scheme 4, the diamine is prepared via a reaction between the pseudo TB precursors and potassium nitrate (KNO₃) in sulfuric acid solution (H₂SO₄) or triluoroacetic anhydride (TFAA) to afford the dinitro compounds, followed by reduction of the dinitro compounds using hydrazine monohydrate (N₂H₄ H₂O) and palladium carbon (Pd/C) to achieve the diamine compounds.

In another embodiment, the pseudo TB tetraamine may generally be synthesized according to the synthetic route illustrated in Scheme 5:

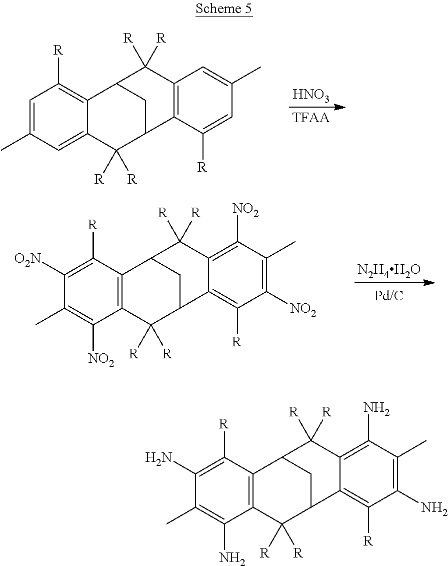

In general, the synthetic route for synthesizing pseudo TB tetraamines is similar to the synthetic route for pseudo TB diamines. In many embodiments, nitric acid and sulfuric acid are used to obtain the intermediate nitro compound.

Non-limiting and non-exhaustive examples of synthetic routes to forming pseudo TB amines are shown in Scheme 6.

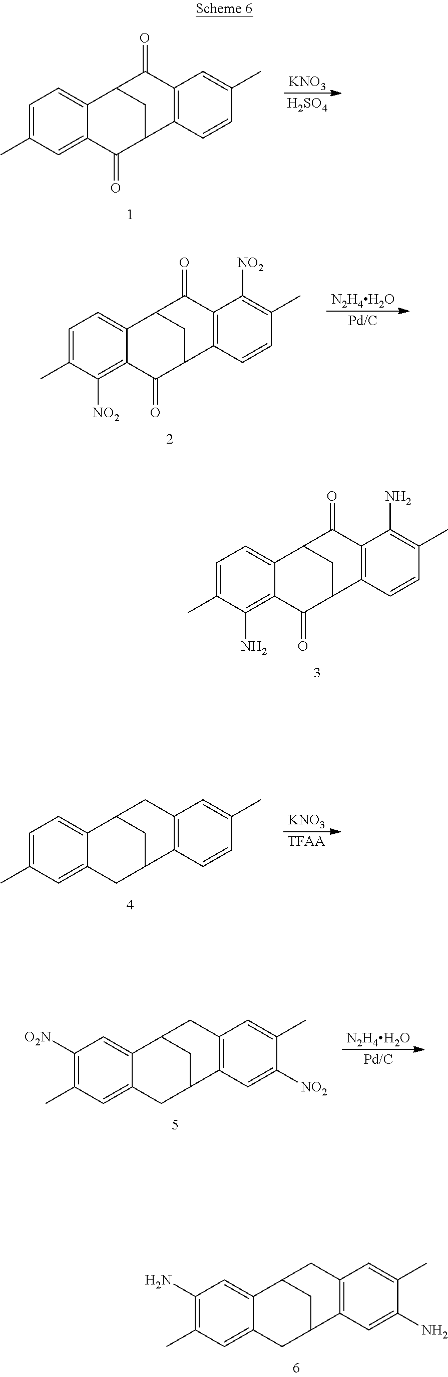

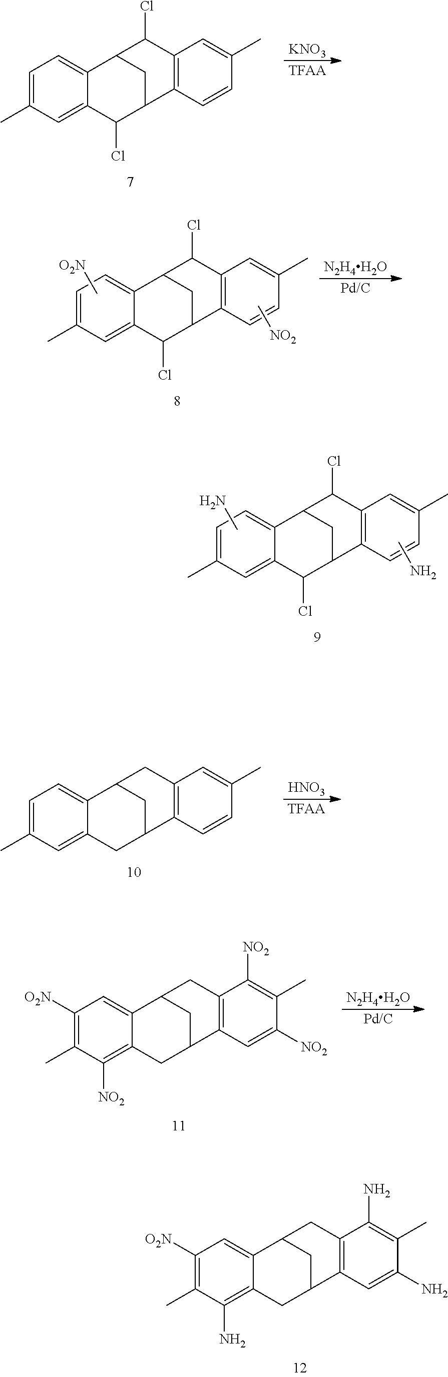

The pseudo TB amine monomers may be used in the synthesis of polymers of intrinsic porosity polyimides (PIM-PI) and network polymers (e.g., network porous polymers). For example, the PIM-PIs may be characterized by the following chemical structure:

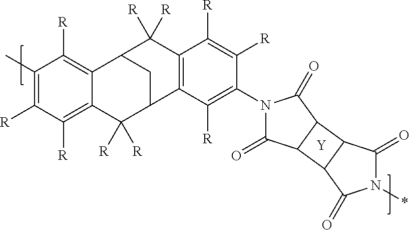

where Y is any dianhydride and/or multianhydride and each R is independently one or more of a hydrogen, a halogen and an alkyl group. The dianhydride and/or multianhydride may be aromatic, cycloaliphatic, and/or aliphatic. The network polymers may be characterized by the following chemical structure:

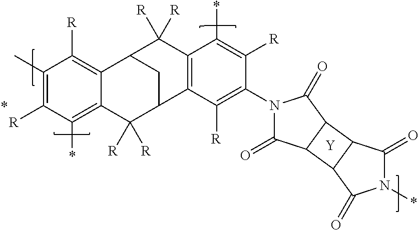

where Y is any dianhydride and/or multianhydride and each R is independently one or more of a hydrogen, a halogen and an alkyl group. The dianhydride and/or multianhydride may be aromatic, cycloaliphatic, and/or aliphatic.

### Methods of Fabricating Microporous Polymers

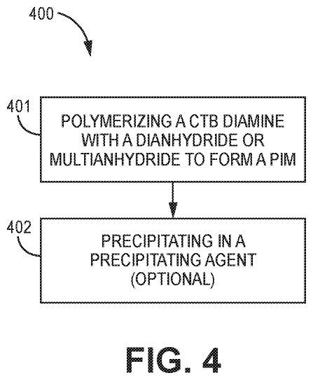

FIG. 4 is a flowchart of a method of fabricating a microporous polymer, according to one or more embodiments of the present disclosure. As shown in FIG. 4, the microporous polymer may be fabricated by polymerizing 401 a pseudo TB amine with an anhydride monomer to form the microporous polymer; and optionally precipitating in a precipitating agent, such as water or methanol. In many embodiments, the microporous polymer is a polymer of intrinsic microporosity polyimide (PIM-PI) or a network polymer (e.g., network porous polymer). The PIM-PI and network polymer may include and/or be characterized by any of the PIM-PIs and network polymers described in the present disclosure.

Polymerizing may include a polycondensation reaction. In many embodiments, polymerizing includes a high-temperature polycondensation reaction. In particular, the polycondensation reaction may occur at gradually increasing temperatures. For example, the polycondensation reaction may occur at gradually increasing temperatures ranging from about room temperature to about 200° C. The ratio of the pseudo TB amine to anhydride monomer may be a 1:1 ratio or a 1:2 ratio. For example, in some embodiments, the polycondensation reaction may proceed between about equimolar amounts of pseudo TB amine and anhydride monomer in a solvent. In other embodiments, the polycondensation reaction may proceed between about non-equimolar amounts of pseudo TB amine and anhydride monomer in a solvent. For example, the ratio of pseudo TB amine to anhydride monomer may be about 1:2. In many embodiments, an equimolar amount of pseudo TB amine and anhydride monomer may be used to prepare PIM-PIs, whereas a 1:2 ratio of pseudo TB amine-anhydride monomer may be used to prepare network polymers. In other embodiments, the desired microporous polymer may be prepared simply by varying the ratio of pseudo TB amine to anhydride monomer.

The pseudo TB amine may include any of the pseudo TB amines of the present disclosure. For example, the pseudo TB amine may include a pseudo TB amine diamine monomer or a pseudo TB tetraamine monomer. In many embodiments, the PIM-PI is prepared from a pseudo TB diamine monomer, and the network polymer is prepared from a pseudo TB tetraamine monomer. In other embodiments, the PIM-PI is prepared from a pseudo TB tetraamine monomer, and the network polymer is prepared from a pseudo TB diamine monomer.

The anhydride monomer may include any anhydride of the present disclosure. For example, the anhydride may be a tetracarboxylic dianhydride monomer characterized by the following chemical structure:

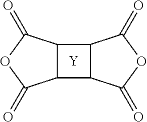

where Y may be characterized by one or more of the following chemical structures:

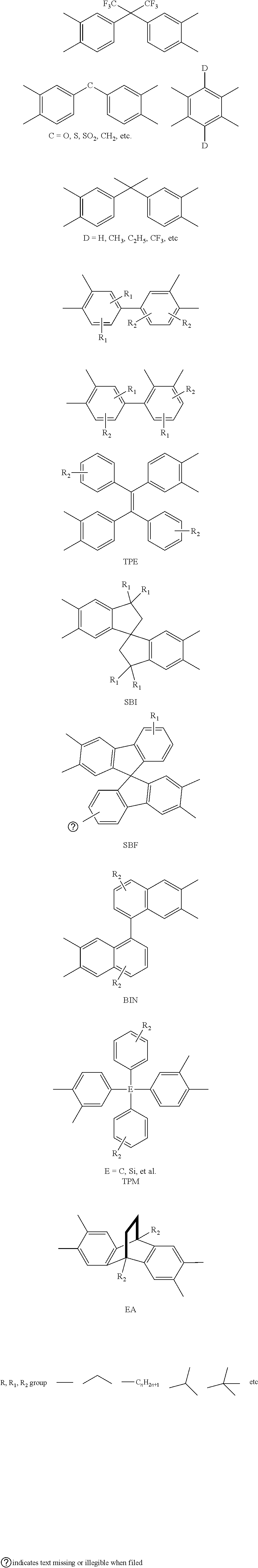

In many embodiments, the anhydride is 4,4′(hexafluoroisopropylidene)-diphthalic anhydride (6FDA). In other embodiments, any of the anhydrides of the present disclosure may be used. For example, any of the anhydrides discussed above with respect to PIM-PIs may be used. Accordingly, that discussion is hereby incorporated by reference in its entirety.

The solvent may include a phenol containing a catalytic amount of an organic compound, wherein the organic compound includes at least one nitrogen. The phenol may include phenols and derivatives thereof. For example, in many embodiments, the phenol is a phenol derivative, such as m-cresol, and the phenol derivatives isomers, such as p-cresol and o-cresol. The organic compound containing at least one nitrogen may include a heterocyclic aromatic organic compound. In many embodiments, the organic compound containing at least one nitrogen is quinoline, as well as derivatives and isomers thereof. For example, the organic compound containing at least one nitrogen may be isoquinoline.

The microporous polymer may be a PIM-PI or microporous network polymer. In embodiments in which the microporous polymer is a PIM-PI, the PIM-PI may be characterized by the following chemical structure:

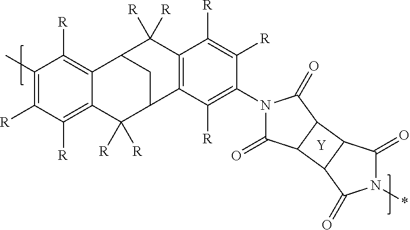

In embodiments in which the microporous polymer is a microporous network polymer, the network polymers may be characterized by the following chemical structure:

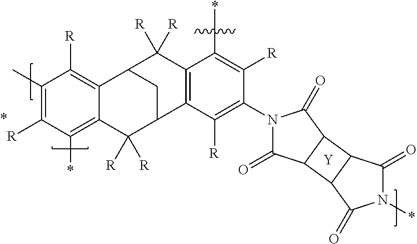

For each of the PIM-PIs and microporous network polymers, Y may include any of the anhydrides (e.g., dianhydrides and/or multianhydrides) of the present disclosure and R may include any of the hydrogen, halogens and alkyl groups of the present disclosure.

The PIM-PIs and network polymers may be formed via similar synthetic routes. Scheme 7 is one example of a synthetic route for preparing PIM-PIs and Scheme 8 is one example of a synthetic route for preparing network polymers:

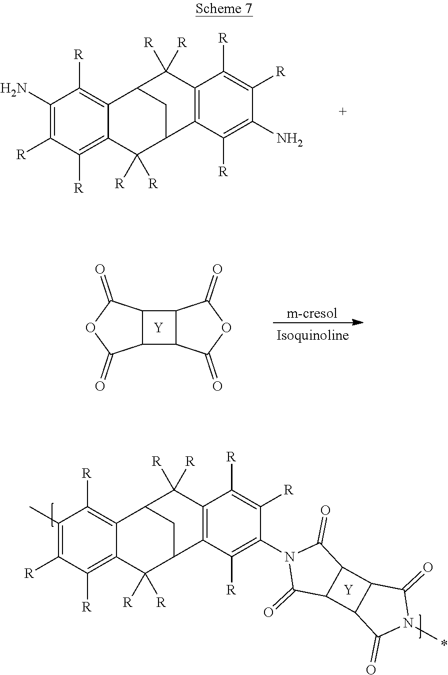

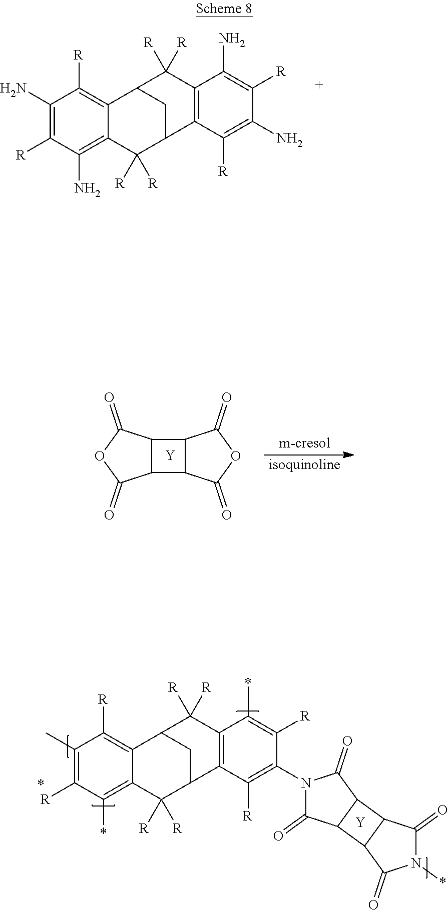

Non-limiting and non-exhaustive examples of synthetic routes to forming PIM-PIs and microporous network polymers are shown in Scheme 9:

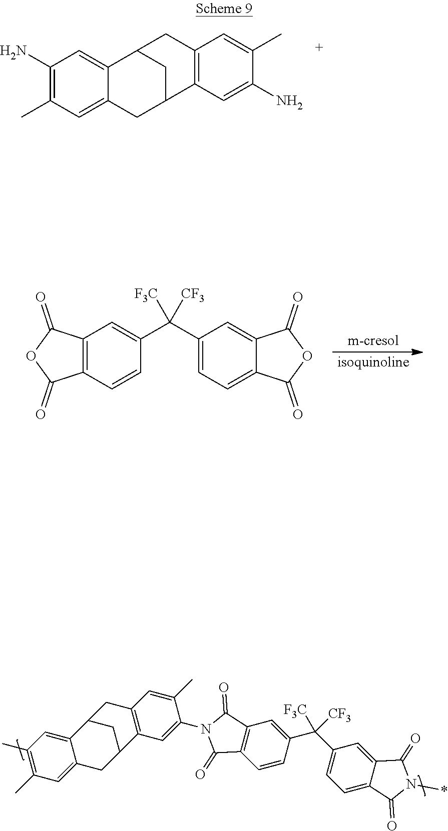

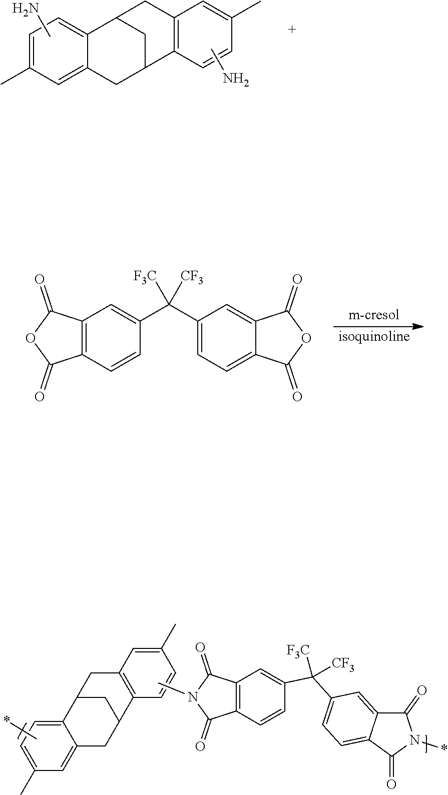

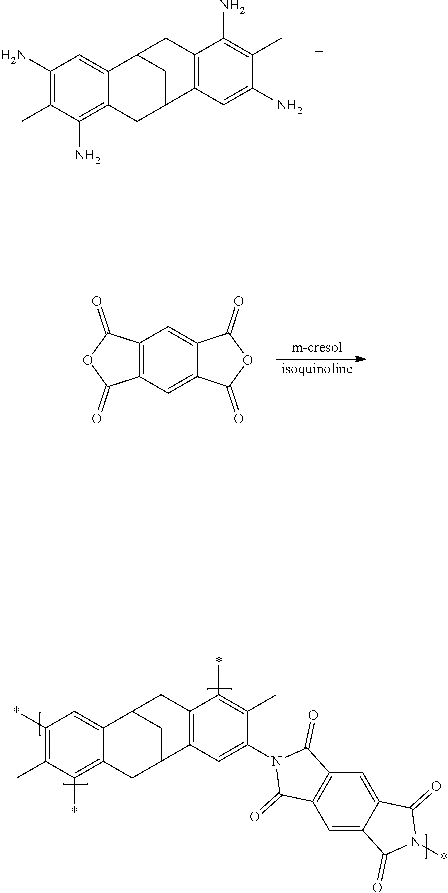

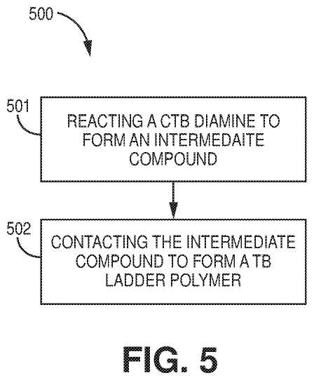

FIG. 5 is a flowchart of a method of forming a ladder polymer of intrinsic porosity, according to one or more embodiments of the present disclosure. At step 501, a pseudo TB amine monomer is reacted with a first solution containing an acidic compound to form an intermediate compound. At step 502, the intermediate compound is contacted with a second solution containing a basic compound to form a ladder polymer of intrinsic porosity. In some embodiments, the method may optionally further include washing with an alcohol (e.g., methanol) and re-precipitating from chloroform in the alcohol (e.g., methanol). The ladder polymer of intrinsic porosity may include and/or be characterized by any of the ladder polymers of intrinsic porosity of the present disclosure.

Reacting may include stirring, mixing, agitating, vibrating, and any other methods of reacting known in the art. The reacting may occur at room temperature for about 48 hours. In many embodiments, the reacting includes stirring at about room temperature for about 48 hours.

The first solution containing an acidic compound may include a solution including trifluoroacetic acid (TFA) and dimethoxymethane (DMM). The second solution containing a basic compound may include ammonium hydroxide. The disclosed first and second solutions and acidic and basic compounds shall not be limiting, as any solution, acidic compound, and basic compound known in the art may be used.

The pseudo TB amine may include any of the pseudo TB amines of the present disclosure. For example, the pseudo TB amine may include a pseudo TB amine diamine monomer or a pseudo TB tetraamine monomer. In many embodiments, the PIM-PI is prepared from a pseudo TB diamine monomer, and the network polymer is prepared from a pseudo TB tetraamine monomer. In other embodiments, the PIM-PI is prepared from a pseudo TB tetraamine monomer, and the network polymer is prepared from a pseudo TB diamine monomer.

The ladder polymers of intrinsic porosity may be characterized by the following chemical structure:

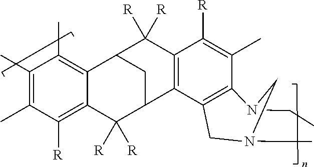

where each R is independently one or more of a hydrogen, halogen and alkyl group. Scheme 10 is one example of a synthetic route for forming ladder polymers of intrinsic porosity:

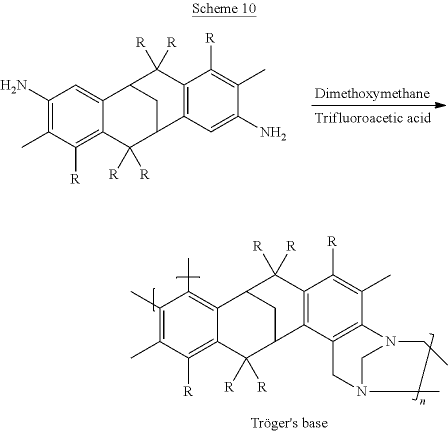

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### Example 1

The following example relates to the synthesis and gas transport properties of a soluble, high molecular weight intrinsically microporous polyimide made from a novel carbocyclic pseudo Tröger base-derived diamine (CTBDA) and 4,4′-(hexafluoroisopropylidene)diphthalic anhydride (6FDA) via high-temperature polycondensation reaction. The polyimides were fully characterized by ¹H NMR, FTIR, GPC, TGA and BET surface area measurements. Moreover, pure-gas permeation data for fresh and aged samples are reported.

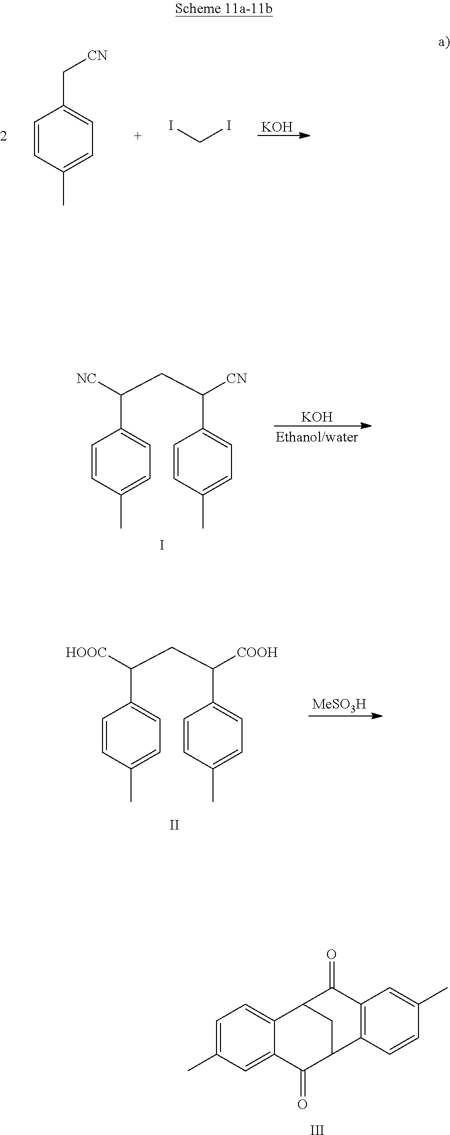

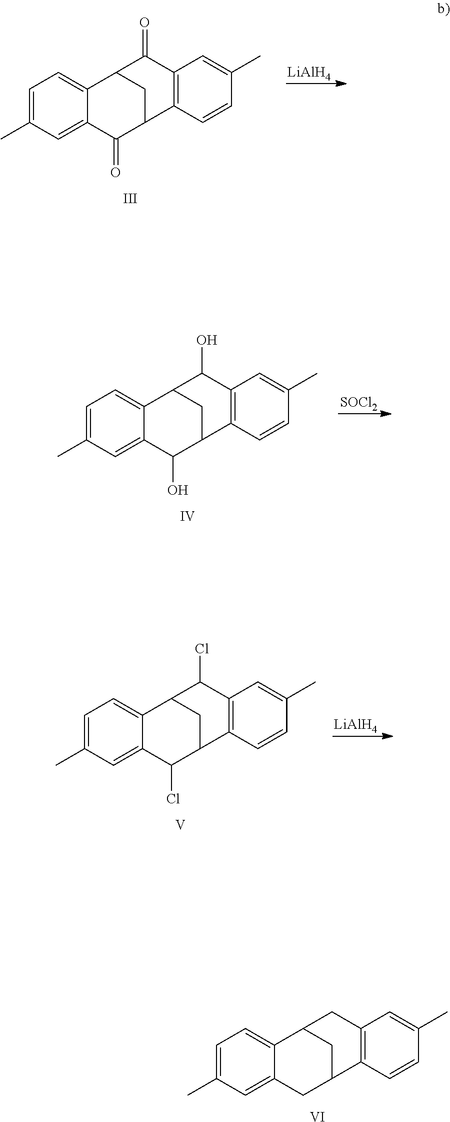

### Synthesis of Pseudo TB Amine Monomers

Synthesis of 2,8-dimethyl-5,11-methanodibenzo[a,e][8]annulene-6,12(5H, 11H)-dione (III) (Scheme 11a). 2,8-dimethyl-5,11-methanodibenzo[a,e][8]annulene-6,12(5H,11H)-dione was prepared. 4-Methylbenzyl cyanide (8 g, 61.0 mmol) and KOH (3.41 g, 61 mmol) were dissolved in diiodomethane (8.3 g, 31 mmol) and heated at 165° C. for 2 hours. The reaction mixture was cooled down and poured into water (200 mL), extracted with dichloromethane (3×50 ml), washed with brine, dried over MgSO₄, and the solvent was removed under vacuum to give meso-phenylpentanedinitrile (I) (8 g), which was hydrolyzed by heating for 18 h at 80° C. in a mixture of ethanol (80 ml) and potassium hydroxide solution (160 ml, 40%). Ethanol was removed under vacuum, and the residue was diluted with water and washed with dichloromethane until the organic phase became colorless. The aqueous phase was acidified to pH<1 by adding concentrated HCl (20 ml) and extracted with ethyl acetate (3×50 ml), dried over MgSO₄ and the solvents were removed under vacuum to give crude meso-phenylpentanedioic acids (II) (6 g). The crude acids were heated at 100° C. for 3 h in methanesulfonic acid (CH₃SO₃H), poured on ice and extracted with ethyl acetate. The organic layers were combined, washed with KOH solution (5 wt. %), dried over MgSO₄, filtered and evaporated to dryness to give crude (III). Purification by silica gel chromatography using dichloromethane/ethyl acetate: 100/1 afforded pure (III) as a white solid (4 g, yield: 64%); mp=182.2° C. ¹H NMR (400 MHz, DMSO-d₆): 7.62 (br s, 2H), 7.4-7.43 (dd, 2H, J=8.8 Hz, 1.2 Hz), 7.3 (d, 2H, J=7.6 Hz), 3.95 (t, 2H, J=2.8 Hz), 2.92 (t, 2H, J=2.8 Hz), 2.27 (s, 6H). ¹³C NMR (100 MHz, DMSO-d₆): 194.7, 138.9, 137.8, 135.7, 129.6, 129.2, 128.6, 127.9, 48.1, 32.0, 21.0.

Synthesis of 2,8-dimethyl-5,6,11,12-tetrahydro-5,1-methanodibenzo[a,e][8]annulene-6,12-diol (IV) (Scheme 11b). 2,8-Dimethyl-5,11-methanodibenzo[a,e][8]annulene-6,12(5H,11H)-dione (III) (2.00 g, 7.24 mmol) was dissolved in THF (100 mL) and then LiAlH₄ (1.1 g, 28.9 mmol) was added in portions. The mixture was stirred at room temperature overnight, then poured on 150 g ice and HCl (6N) was added. The solution was extracted with dichloromethane (3×50 ml), dried over MgSO₄, filtered and the solvent was removed by vacuum. The resulting yellowish solid was washed using a n-hexane/dichloromethane mixture (1:1) to afford an off-white powder (1.42 g, yield: 71%) as a final product; mp=215.6° C. ¹H NMR (400 MHz, CDCl₃): 7.40 (s, 2H), 7.18 (d, 2H, J=8 Hz), 7.03 (d, 2H, J=7.6 Hz), 5.02 (d, 2H, J=5.6 Hz), 3.3 (m, 2H), 2.4 (t, 2H, J=3.2 Hz), 2.29 (s, 6H), 1.66 (s, 2H). ¹³C NMR (100 MHz, CDCl₃): 139.4, 137.4, 130.9, 129.9, 128.2, 127.8, 72.6, 39.1, 29.5, 21.2.

Synthesis of 6,12-dichloro-2,8-dimethyl-5,6,11,12-tetrahydro-5,1-methanodibenzo[a,e][8]annulene (V) (Scheme 11b). 2,8-Dimethyl-5,6,11,12-tetrahydro-5,11-methanodibenzo[a,e][8]annulene-6,12-diol (v) (2 g, 7.13 mmol) was suspended in SOCl₂ (30 mL) and 0.3 ml DMF were added. The solution was refluxed overnight and SOCl₂ was removed by vacuum. The collected product was dried at 100° C. for 3 h. The resulting product (2.1 g, yield: 93%) was obtained as an off-white solid; mp=191.0° C. ¹H NMR (400 MHz, CDCl₃): 6.19 (d, 2H, J=8 Hz), 7.08 (d, 2H, J=7.6 Hz), 7.02 (s, 2H), 5.05 (d, 2H, J=1.6 Hz), 3.54 (m, 2H), 2.67 (t, 2H, J=2.8 Hz), 2.26 (s, 6H). ¹³C NMR (100 MHz, CDCl₃): 137.9, 133.8, 133.6, 131.6, 130, 129.3, 62.2, 40.9, 21.0, 18.7.

Synthesis of 2,8-dimethyl-5,6,11,12-tetrahydro-5,11-methanodibenzo[a,e][8]annulene (VI) (Scheme 11b). 6,12-Dichloro-2,8-dimethyl-5,6,11,12-tetrahydro-5,11-methanodibenzo[a,e][8]annulene (V) (5 g, 15.8 mmol) was dissolved in THF (250 ml) and LiAlH₄ (2.4 g, 63 mmol) was added in portions over 30 minutes. The reaction was refluxed overnight and the resulting mixture was then poured on ice (200 g) and HCl (6N, 100 ml) was added. The solution was extracted with dichloromethane three times, dried over MgSO₄, filtered and then the solvent was removed by rota-evaporation. The resulted light orange powder was washed by n-hexane/DCM: 4/1 to afford VI (3 g, yield: 76%) as a white powder product; mp=109.5° C. ¹H NMR (400 MHz, CDCl₃): 7.11 (d, 2H, J=7.6 Hz), 6.94 (d, 2H, J=7.6 Hz), 6.78 (s, 2H), 3.29 (m, 2H), 3.25 (d, 2H, J=5.2 Hz), 2.81 (d, 2H, J=16 Hz), 2.24 (s, 6H), 2.13 (t, 2H, J=2.8 Hz). ¹³C NMR (100 MHz, CDCl₃): 138.2, 135.4, 134.4, 129.9, 128.7, 126.7, 39.5, 32.56, 29.1, 21.0.

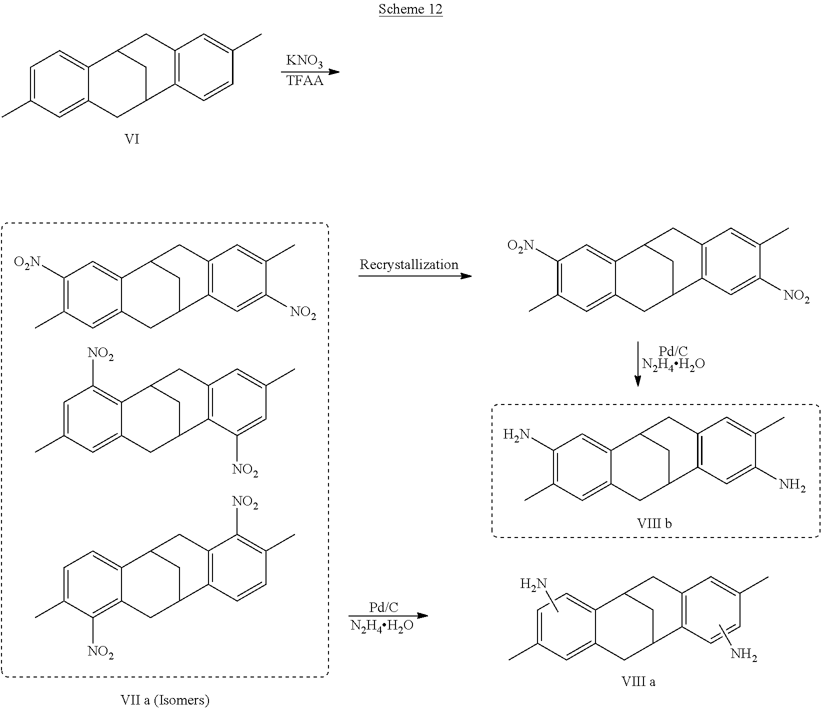

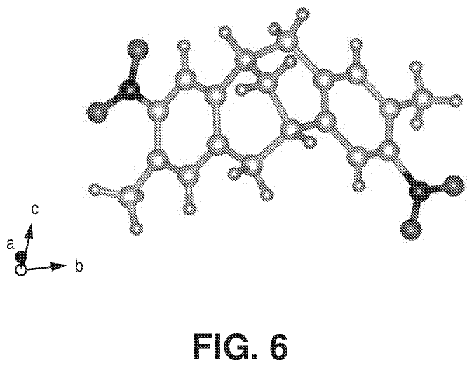

Synthesis of 2,8-dimethyl-1,7(4,10)(3,9)-dinitro-5,6,11,12-tetrahydro-5,11-methanodibenzo[a,e][8]annulene (VII a) and 2,8-dimethyl-3,9-dinitro-5,6,11,12-tetrahydro-5,11-methanodibenzo[a,e][8]annulene (VII b) (Scheme 12). 2,8-Dimethyl-5,6,11,12-tetrahydro-5,11-methanodibenzo[a,e][8]annulene (VI) (1.25 g, 5 mmol) was dissolved in 50 ml acetonitrile (CH₃CN) followed by the addition of KNO₃ (1.12 g, 11.1 mmol) and then trifluoroacetic anhydride (TFAA) (5.2 ml, 35.7 mmol) was added dropwise. After stirring for 1 hour at room temperature the reaction was poured on ice and then extracted with dichloromethane (DCM). The crude product was purified by silica gel column chromatography using DCM/n-hexane: 1/1 as an eluent. The product was obtained as a yellow powder (0.8 g, yield: 47%); mp=224.5° C. ¹H NMR and ¹³C NMR showed that the product contained three isomers. Recrystallization was performed to obtain only one isomer as a light yellow powder. The structure of the pure isomer was confirmed by single-crystal XRD (FIG. 6). The crystallographic data for VII are deposited in the Cambridge Structural Database (CCDC 1545077). ¹H NMR (400 MHz, CDCl₃) ppm: 7.90 (s, 2H), 6.94 (s, 2H), 3.45 (m, 2H), 3.32 (dd, 2H, J=12 Hz), 2.91 (d, 2H, J=17.2 Hz), 2.49 (s, 6H), 2.19 (t, 2H, J=2.8 Hz). ¹³C NMR (100 MHz, CDCl₃): 147.3, 140.3, 139.4, 133.7, 131.65, 125.2, 39.3, 31.9, 28.2, 20.3.

Synthesis of 2,8-dimethyl-3,9-diamine-5,6,11,12-tetrahydro-5,11-methanodibenzo[a,e][8]annulene (VIII b, CTBDA) (Scheme 12). 2,8-Dimethyl-3,9-dinitro-5,6,11,12-tetrahydro-5,11-methanodibenzo[a,e][8]annulene (VII b) (0.4 g, 1.2 mmol) was suspended in 20 ml ethanol followed by the addition of Pd/C (0.2 g) and 2 ml N₂H₄H₂O. The mixture was refluxed for 3 hours under nitrogen and then cooled down to room temperature, precipitated in water and filtrated. The white solid was dried in the vacuum oven for 24 h at 60° C. (0.26 g, yield: 80%); mp=192.2° C. ¹H NMR (400 MHz, DMSO-d6) ppm: 6.44 (d, 2H, J=4 Hz), 6.40 (d, 2H, J=3.6 Hz), 4.46 (br s, 4H), 3.1 (d, 2H, J=18 Hz), 3.0 (m, 4H), 2.47 (m, 2H), 1.9 (s, 6H). ¹³C NMR (100 MHz, DMSO-d6): 144.6, 139.3, 130.5, 122.4, 120.2, 114.3, 39.0, 32.8, 18.0, 17.4. The same synthetic procedure was applied to the produce the 2,8-dimethyl-(3,9)(1,7)(4,10)-diamine-5,6,11,12-tetrahydro-5,11-methanodibenzo[a,e][8]annulene (VIII a, iCTBDA).

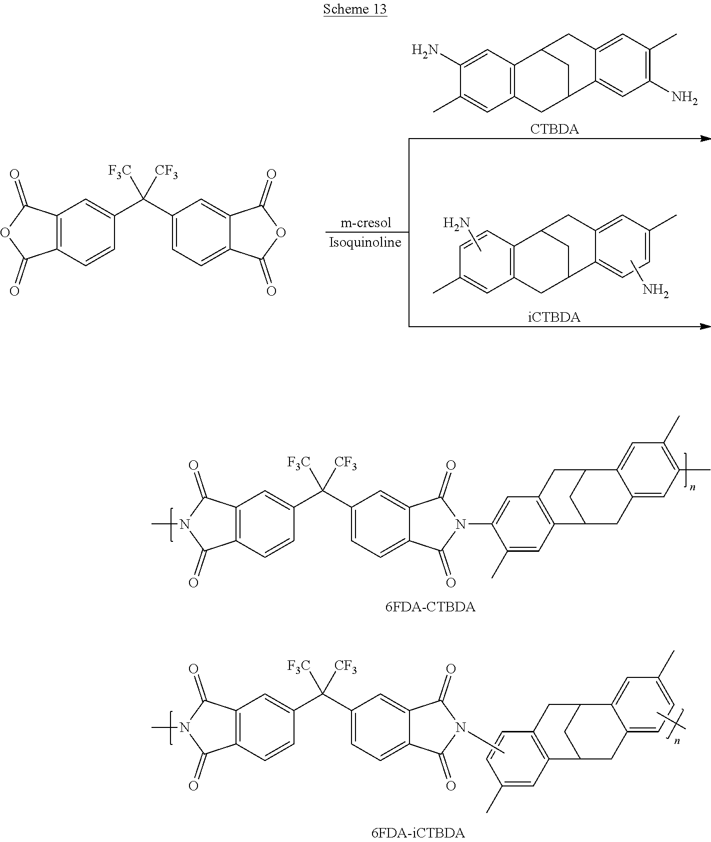

Synthesis of polyimides (Scheme 13). To a dry 25 ml reaction tube equipped with a Dean-Stark trap, nitrogen inlet and outlet, and reflux condenser were added the diamine (VIII a, VIII b) (1.0 mmol), equimolar amount of the dianhydride monomer (6FDA) (1.0 mmol) and isoquinoline (0.1 ml) in m-cresol (2 ml). The reaction mixture was stirred at room temperature for 1 h, and the temperature was then raised gradually to 200° C. and kept at that temperature for 4 h under a steady flow of nitrogen. Fibrous polyimide was obtained by the dropwise addition of the polymer solution to an excess of methanol (300 ml). The resulting solid fibers were filtered off and the polymer was purified by re-precipitation from chloroform solution into methanol and dried at 120° C. in a vacuum oven for 24 h to give about 90% yield.

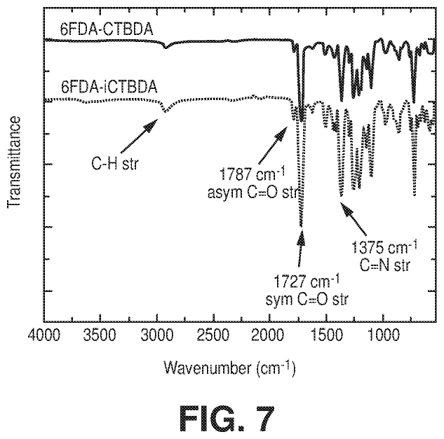

Synthesis of 6FDA-CTBDA and 6FDA-iCTBDA. Following the above general procedure 6FDA-CTBDA and 6FDA-iCTBDA were prepared from 6FDA dianhydride and TB diamine VIII a or XIII b, respectively, and obtained as off-white powder (˜80-90% yield). ¹H NMR (400 MHz, DMSO-d6, δ): 2.02 (br s, 6H), 2.68 (br m, 2H), 3.25-3.34 (br m, 6H), 6.98 (br s, 2H), 7.24 (br s, 2H), 7.79 (br s, 2H), 7.96 (br s, 2H), 8.15 (br s, 2H). FT-IR (Powder, ν, cm⁻¹): 1785 (C═O asym), 1724 (C═O sym, str), 1367 (C—N, str), 722 (imide ring deformation); BET surface area=587 [562] m² g⁻¹; GPC (DMF): Mₙ=100,000 [85,000] g mol⁻¹, Mw=164,000 [155,000] g mol-; PDI=1.64 [1.82]. TGA: T^{d,5%} at ˜490 [490]° C. Numbers in brackets are for 6FDA-iCTBDA. FIG. 7 shows FT-IR spectra of 6FDA-CTBDA and 6FDA-iCTBDA polyimides, according to one or more embodiments of the present disclosure.f

Polymer Film Preparation.

6FDA-CTBDA solutions in chloroform (2-3% w/v, g/ml) were filtered through 0.45 μm polypropylene filters and clear isotropic films were obtained by slow evaporation of the solvent at room temperature from a leveled petri dish. The dry films were soaked for 24 h in methanol to remove any residual solvent traces, air-dried and then heated at 120° C. for 24 h in a vacuum oven. TGA was used to confirm complete removal of solvent traces. Films with thickness of ˜40 μm were used for gas permeability measurements.

Gas sorption measurements. A Micromeritics ASAP 2020 gas sorption analyzer equipped with a micropore upgrade was used to measure the BET surface area of 6FDA-CTBDA. Nitrogen sorption measurements were performed at −196° C. up to 1 bar. Analysis of the pore size distributions was performed using the NLDFT (Non-Local Density Functional Theory) model using N₂ sorption isotherms for carbon slit pore geometry provided by ASAP 2020 version 4.02 software.

Carbon dioxide and methane sorption in 6FDA-CTBDA was measured at 35° C. up to ˜15 bar using a Hiden Intelligent Gravimetric Analyzer (IGA-003, Hiden Isochema, UK). After drying a polymer film sample (˜40-50 mg) under vacuum at 80° C. for 2 days, it was mounted in the sorption apparatus and degassed under high vacuum (<10⁻⁷ mbar) at 35° C. until constant sample weight readings were obtained before beginning collection of the isotherm data. Then, gas was introduced in the sample chamber by a stepwise pressure ramp of 100 mbar/min until a desired pressure was reached. After equilibrium weight uptake was recorded, the next pressure point was set, and this process was continued until the complete isotherm was determined.

Gas permeation measurements. The pure-gas permeability of H₂, N₂, O₂, CH₄ and CO₂ was measured at 35° C. and 2 bar via the constant-volume/variable pressure method and calculated by:

$$P = {10}^{10} \frac{{V}_{d} l}{{P}_{up} ART} \frac{d p}{d t}$$

where P is the permeability coefficient in Barrers (1 Barrer=10⁻¹⁰ cm³ (STP) cm cm⁻² s⁻¹ cmHg⁻¹), V is the calibrated volume of the downstream gas reservoir (cm³), L is the film thickness (cm), A is the effective membrane area (cm²), R is the gas constant (0.278 cm³ cmHg cm⁻³ (STP) K⁻¹), T is the operating temperature (K), pᵤₚ is the upstream pressure (cmHg), and dp/dt is the steady-state permeate-side pressure increase (cmHg s⁻¹). Gas permeation in polymers follows a solution/diffusion transport mechanism according to: P=D×S, where D is the apparent diffusion coefficient (cm² s⁻¹) and S is the solubility coefficient (cm³ (STP) cm⁻³ cmHg⁻¹). Gas solubilities of CO₂ and CH₄ were measured gravimetrically at 35° C. up to ˜15 bar and then diffusion coefficients were calculated from D=P/S.

The ideal pure-gas selectivity for a gas pair is given by the following relationship:

$${α}_{A / B} = \frac{{P}_{A}}{{P}_{B}} = \frac{{D}_{A}}{{D}_{B}} \times \frac{{S}_{A}}{{S}_{B}}$$

where α_{A/B} is the permselectivity of gas A over gas B which can be factored into the diffusion (D_{A}/D_{B}) and solubility (S_{A}/S_{B}) selectivity, respectively.

The polyimides were further characterized by GPC, TGA, and BET surface area (Table 1). The carbocyclic pseudo CTBDA-based polyimides showed high average molecular weights (M_{w}˜155,000-167,000 g mol⁻¹) and narrow polydispersity index of ˜1.6-1.8.

TABLE 1
Physical properties of 6FDA-CTBDA and 6FDA-iCTBDA polyimides.

| | | | | | | BET |
|---|---|---|---|---|---|---|
| | | | | | | surface |
| | M_{w} | Mₙ | PDI | T_{d,5%} | Density | area |
| Polymer | (g mol⁻¹) | (g mol⁻¹) | (−) | (° C.) | (g mol⁻³) | (m²g⁻¹⁾ |
| 6FDA- | 164,000 | 100,000 | 1.64 | 490 | 1.26 | 587 |
| CTBDA | | | | | | |
| 6FDA- | 155,000 | 85,000 | 1.82 | 490 | 1.30 | 562 |
| iCTBDA | | | | | | |

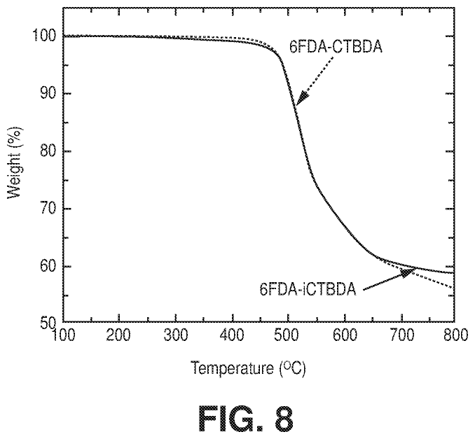

The polyimides showed excellent solubility in common organic solvents, such as CHCl₃, THF, DMF, DMAc, NMP, and DMSO. The 6FDA-CTBDA polyimides exhibited high thermal stability with T_{d,5%} of ˜490 and 450° C., respectively, as determined by TGA in nitrogen atmosphere (FIG. 8).

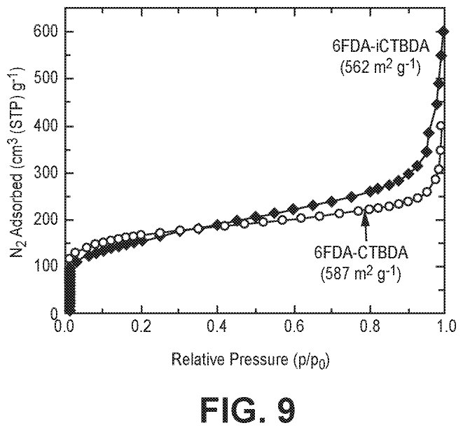

Nitrogen adsorption isotherms of 6FDA-CTBDA and 6FDA-iCTBDA measured at −196° C. up to 1 bar are shown in FIG. 9. High nitrogen uptake was evident at low relative pressure, indicating the presence of intrinsic microporosity in the polyimides. The BET surface areas of 6FDA-CTBDA (587 m² g⁻¹) and 6FDA-iCTBDA (562 m² g⁻¹) were practically identical within experimental error.

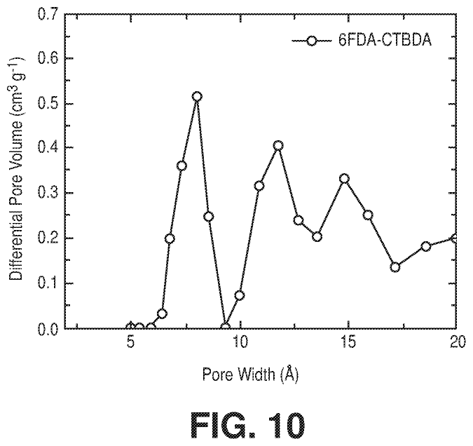

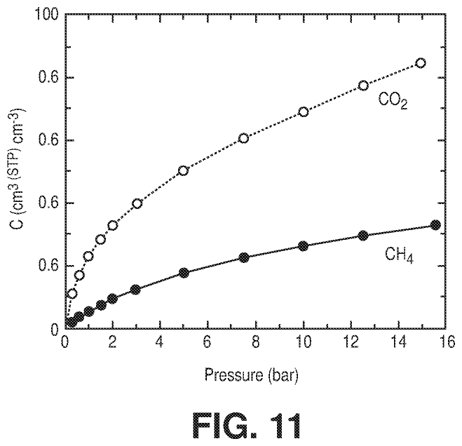

The NLDFT-derived pore size distribution for 6FDA-CTBDA calculated based on their N₂ adsorption isotherms are shown in FIG. 10 The polyimide displayed bimodal pore size distributions with pores in the ultra-microporous range (<7 Å) and a large fraction of micropores in the range of 10-20 Å. FIG. 11 is graphical view of CO₂ and CH₄ sorption isotherms measured gravimetrically at 35° C. for 6FDA-CTBDA, according to one or more embodiments of the present disclosure.

Gas transport properties. Pure-gas permeation experiments were performed at 2 bar and 35° C. on fresh and 60-day aged samples of 6FDA-CTBDA and 6FDA-iCTBDA. Both CTBDA-derived polyimides exhibited high permeabilities and moderate selectivities, as shown in Table 2. The gas permeabilities of the polyimides followed the order: H₂>CO₂>O₂>N₂>CH₄, a trend that is typically observed for moderately microporous PIM-PIs. The gas permeabilities of the two CTBDA-based polyimides were similar; for example the CO₂ permeabilities of fresh 6FDA-CTBDA and 6FDA-iCTBDA films were 291 and 230 Barrer, respectively, with identical CO₂CH₄ selectivity of 25. This result indicates that isomerism in the CTB moiety of the 6FDA polyimides had only a small effect on their gas permeation properties. Physical aging of the 6FDA-CTBDA film over 60 days resulted in ˜30-40% decrease in permeabilities with small increase in selectivities. Compared to commercial membrane materials for CO₂/CH₄ separation, such as cellulose triacetate (CTA), aged 6FDA-CTBDA showed commendable performance with ˜30-fold higher CO₂ permeability of 201 Barrer (vs. 6.6 Barrer for CTA) and similar CO₂/CH₄ selectivity of 28 (vs. 32 for CTA).

TABLE 2
Pure-gas permeabilities and selectivities for 6FDA-CTBDA and 6FDA-
iCTBDA (2 bar; 35° C.; film thickness ~40 μm).

| | Pure-gas permeability (Barrer) | | | | | Selectivity (α) | | |
|---|---|---|---|---|---|---|---|---|
| Polymer | H₂ | N₂ | O₂ | CH₄ | CO₂ | CO₂/CH₄ | H₂/CH₄ | O₂/N₂ |
| 6FDA- | 375 | 14.8 | 56 | 11.6 | 291 | 25 | 32 | 3.8 |
| CTBDA | | | | | | | | |
| 6FDA- | 286 | 10.3 | 43 | 7.2 | 201 | 28 | 40 | 4.2 |
| CTBDA* | | | | | | | | |
| 6FDA- | 313 | 12.2 | 49 | 8.9 | 230 | 25 | 35 | 3.9 |
| iCTBDA | | | | | | | | |

*60 days aged sample.

In the present invention, CO₂ and CH₄ sorption isotherms of 6FDA-CTBDA was measured directly by gravimetric gas sorption at 35° C. up to ˜15 bar. The CO₂ and CH₄ solubility coefficients measured at 2 bar are shown in Table 3. The CO₂/CH₄ solubility selectivity of 6FDA-CTBA was 3.5.

### Example 2

Synthesis of 2,8-dimethyl-3,9-dinitro-5,6,11,12-tetrahydro-5,11-methanodibenzo[a,e][8]annulene (5). The dinitro compound can be prepared via a reaction between 2,8-dimethyl-5,6,11,12-tetrahydro-5,11-methanodibenzo[a,e][8]annulene (VI) (4) (4 mmol) and potassium nitrate (KNO₃) (8.2 mmol) in 8 ml of trifluoroacetic anhydride solution (TFAA). The obtained dinitro compound was purified by using silica gel column chromatography using 1/1 dichloromethane/hexane. A light yellow product was obtained (yield=47%). NMR spectroscopy showed that the dinitro compound was obtained as three isomers. To afford a single isomer the product was recrystallized in methanol, and the resulting solid was filtered and dried in an oven at 60° C. for 24 hours (See Scheme 6). ¹H NMR (400 MHz, CDCl₃) ppm: 7.90 (s, 2H), 6.94 (s, 2H), 3.45 (m, 2H), 3.32 (dd, 2H, J=12 Hz), 2.91 (d, 2H, J=17.2 Hz), 2.49 (s, 6H), 2.19 (t, 2H, J=2.8 Hz). ¹³C NMR (100 MHz, CDCl₃): 147.3, 140.3, 139.4, 133.7, 125.2, 39.3, 31.9, 28.2, 20.3.

Synthesis of 2,8-dimethyl-5,6,11,12-tetrahydro-5,11-methanodibenzo[a,e][8]annulene-3,9-diamine (6). 2,8-dimethyl-3,9-dinitro-5,6,11,12-tetrahydro-5,11-methanodibenzo[a,e][8]annulene (VII b) (5) (1.5 mmol) was suspended in 20 ml ethanol followed by the addition of Pd/C (0.25 g) and N₂H₄H₂O (2.5 ml). The obtained mixture was refluxed for 3 hours under nitrogen. The system was cooled down to room temperature and precipitated in water and then filtrated. A white solid was obtained with 80% yield. The solid was placed in the vacuum oven for 24 hours at 60° C. (See Scheme 6). ¹H NMR (400 MHz, DMSO-d6) ppm: 6.44 (d, 2H, J=4 Hz), 6.40 (d, 2H, J=3.6 Hz), 4.46 (br s, 4H), 3.1 (d, 2H, J=18 Hz), 3.0 (m, 4H), 2.47 (m, 2H), 1.9 (s, 6H). ¹³C NMR (100 MHz, DMSO-d6): 144.6, 139.3, 130.5, 122.4, 120.2, 114.3, 39.0, 32.8, 18.0, 17.4.

Synthesis of network polymers. To a dry 50 ml reaction tube equipped with a Dean-Stark trap, nitrogen inlet and outlet, and reflux condenser were added 2,8-dimethyl-5,6,11,12-tetrahydro-5,11-methanodibenzo[a,e][8]annulene-1,3,7,9-tetraamine (1.0 mmol), equimolar amount of pyromellitic dianhydrdie PMDA (2.0 mmol) and isoquinoline (0.1 ml) in m-cresol (25 ml). The reaction mixture was stirred at 0° C. for 3 hours followed by 12 hours at room temperature and then the temperature was raised gradually to 200° C. and kept at that temperature for 8 h under steady flow of nitrogen. The obtained precipitation was collected by filtration and washed by tetrahydrofuran (THF) and acetone, then washed by hot methanol for 12 hours using soxhlet extraction. The resulting solid was filtered and dried in an oven at 120° C. over 48 hours to give 50% yield of network polymer (Scheme 9).

Synthesis of ladder polymers. Synthesis of Pseudo Tröger's base/Tröger's base. To a dry 25 ml reaction tube equipped with a Dean-Stark trap, nitrogen inlet and outlet, and reflux condenser were added 2,8-dimethyl-5,6,11,12-tetrahydro-5,11-methanodibenzo[a,e][8]annulene-3,9-diamine (CTBDA) (1.0 mmol) to a solution of trifluoroacetic acid (TFA) followed by the addition of dimethoxymethane (DMM) at 0° C. The reaction was stirred for 48 hours at room temperature, then ammonium hydroxide solution was added to afford the Tröger's base ladder polymer. The obtained powder was washed with methanol and re-precipitated from chloroform in methanol (Scheme 10).

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A carbocyclic pseudo Tröger's base (CTB) diamine, comprising:
a pseudo Tröger's base (PTB) diamine characterized by the following chemical structure: 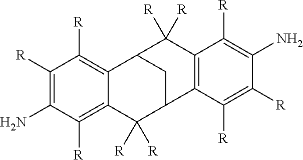
where each R is independently selected from hydrogen, halogens, and alkyl groups.

2. The diamine of claim 1, wherein the halogens are selected from fluorine, chlorine, bromine, and iodine.

3. The diamine of claim 1, wherein the alkyl groups are selected from methyl, ethyl, propyl, isopropyl, butyl, and iso-butyl.

4. The diamine of claim 1, wherein the diamine is characterized by one of the following structures:

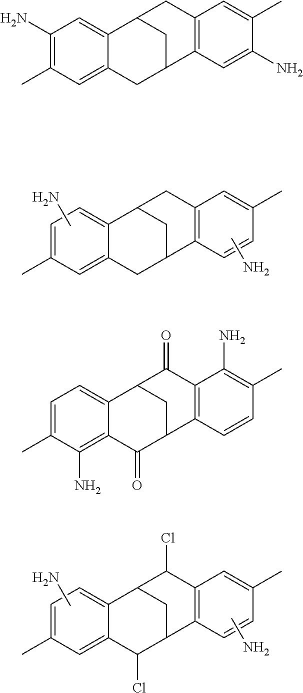

5. A polyimide, comprising:
a polyimide characterized by the following chemical structure: 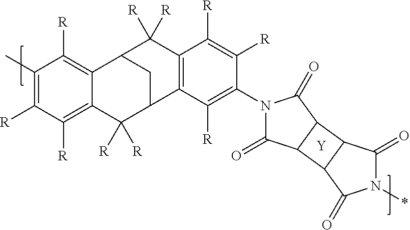
where Y is any dianhydride or multianhydride and each R is independently selected from hydrogen, halogens, and alkyl groups.

6. The polyimide of claim 5, wherein the dianhydride and/or multianhydride is a tetracarboxylic dianhydride monomer characterized by the following chemical structure: 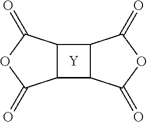
where Y is characterized by one of the following chemical structures:

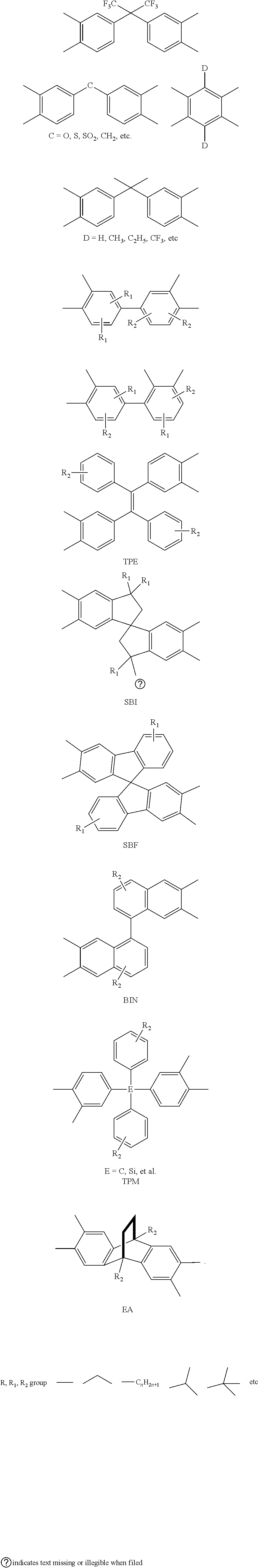

7. The polyimide of claim 5, wherein the polyimide is characterized by one of the following chemical structures:

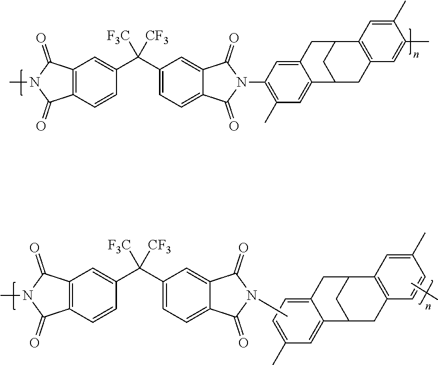

8. A pseudo Tröger's base (CTB) tetraamine, comprising:
a CTB tetraamine characterized by the following chemical structure: 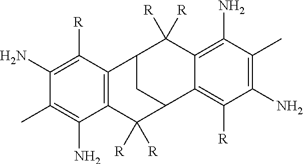
where each R is independently selected from hydrogen, halogens, and alkyl groups.

9. The tetraamine of claim 8, wherein the tetraamine is characterized by the following chemical structure:

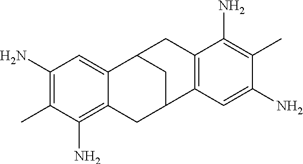 [SMILES: Cc1c(N)cc2c(c1N)CC1CC2Cc2c1cc(N)c(C)c2N]

10. A network porous polymer, comprising:
a network porous polymer characterized by the following chemical structure: 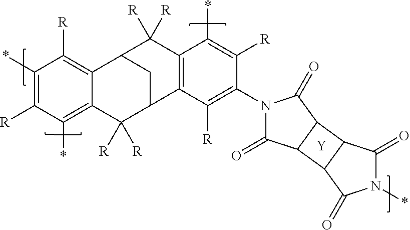
where Y is any dianhydride or multianhydride and each R is independently selected from hydrogen, halogens, and alkyl groups.

11. The polymer of claim 10, wherein the network porous polymer is characterized by the following chemical structure:

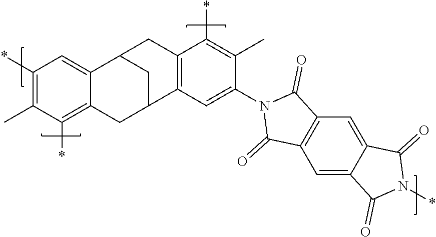

12. A Tröger's base ladder polymer, comprising:
a Tröger's base ladder polymer characterized by the following chemical structure: 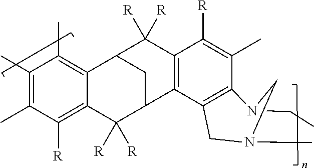
where each R is independently selected from hydrogen, halogens, and alkyl groups.

13. A method of separating chemical species in a fluid composition, comprising:
contacting a microporous polymer membrane with a fluid composition including at least two chemical species; and
capturing at least one of the chemical species from the fluid composition;
wherein the microporous polymer membrane includes a monomer characterized by one of the following chemical structures: 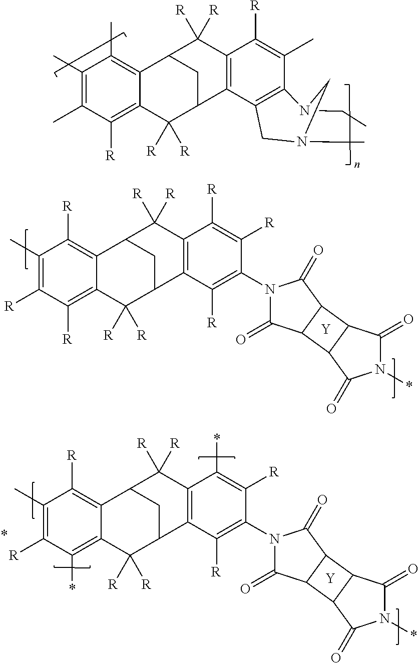
where Y is any anhydride and each R is independently selected from hydrogen, halogens, and alkyl groups.

14. The method of claim 13, wherein contacting includes one or more of feeding, flowing, and passing.

15. The method of claim 13, wherein the chemical species of the fluid composition includes one or more of O₂, N₂, H₂, He, CO₂, C₁₊ hydrocarbons, olefins, paraffins, n-butane, iso-butane, butenes, and xylene isomers.

16. The method of claim 13, wherein capturing includes removing one or more chemical species from the bulk fluid composition.

17. The method of claim 13, wherein the captured chemical species include one or more of O₂, N₂, H₂, He, CO₂, C₁₊ hydrocarbons, olefins, paraffins, n-butane, iso-butane, butenes, and xylene isomers.

18. The method of claim 13, wherein the microporous polymer membrane is used for one or more of the following separations: O₂/N₂, H₂/N₂, H₂/C₁₊ hydrocarbons, He/C₁₊ hydrocarbons, CO₂/C₁₊ hydrocarbons; CO₂/N₂, olefins/paraffins, n-butane/iso-butane, n-butane/butenes, and xylene isomers.

19. The method of claim 13, wherein Y is a dianhydride.

20. The method of claim 13, wherein Y is a multianhydride.
